(19) United States
(12) Patent Application Publication
(10) Pub. No.: US 2005/0266416 A1
(43) Pub. Date: Dec. 1, 2005

(54) Molecular nanomotor

(75) Inventor: Peixuan Guo, West Lafayette, IN (US)

Correspondence Address: MUETING, RAASCH & GEBHARDT, P.A., P.O. BOX 581415, MINNEAPOLIS, MN, 55458, US

(73) Assignee: Purdue Research Foundation, West Lafayette, IN (US)

(21) Appl. No.: 10/938,085

(22) Filed: Sep. 10, 2004

Related U.S. Application Data
(63) Continuation-in-part of application No. 10/699,715, filed on Nov. 3, 2003.
Application No. 10/699,715 is a continuation-in-part of application No. 10/660,132, now abandoned.
(60) Provisional application No. 60/582,661, filed on Jun. 24, 2004.
(60) Provisional application No. 60/411,808, filed on Sep. 18, 2002.
(60) Provisional application No. 60/501,931, filed on Sep. 11, 2003.

(51) Int. Cl. C12Q 1/68; C12N 9/22; C12M 1/34

(52) U.S. Cl. 435/6; 435/199; 435/287.2

(57) ABSTRACT

A molecular nanomotor useful for translocating polynucleotides. The nanomotor is a multimolecular complex fueled by ATP hydrolysis. One of the motor components is an ATP-binding RNA molecule that participates in ATPase activity.

This application is a continuation-in-part application of U.S. patent application Ser. No. 10/699,715, filed Nov. 3, 2003, which is a continuation-in-part application of U.S. patent application Ser. No. 10/660,132, filed Sep. 11, 2003, now abandoned, which claims the benefit of U.S. Provisional Patent Application Ser. No. 60/411,808, filed Sep. 18, 2002; this application further claims the benefit of U.S. Provisional Patent Applications Ser. No. 60/501,931, filed Sep. 11, 2003, and Ser. No. 60/582,661, filed Jun. 24, 2004. Each of these applications is incorporated herein by reference in its entirety.

This application further fully incorporates by reference international patent publications PCT WO 02/16596, published Feb. 28, 2002; U.S. Pat. Publ. 20040157304, published Aug. 12, 2004; and U.S. Pat. Publ. 20040126771, published Jul. 1, 2004.

### STATEMENT OF GOVERNMENT RIGHTS

This invention was made with government support under grants from the National Institutes of Health, Grant Nos. GM59944, and GM60529, and from the National Science Foundation, Grant No. MCB9723923. The U.S. Government has certain rights in this invention.

### BACKGROUND OF THE INVENTION

Nanotechnology refers to the study of the interaction of components on the atomic and molecular scale. At the nanoscale, the physical, chemical, and biological properties of materials may differ fundamentally from the bulk properties of the materials leading to unexpected results because of variations on the quantum mechanical properties of atomic interactions.

Current research efforts are directed toward the characterization, manipulation, modification, control, creation, and/or assembly of organized materials on the nanoscale level (A. Modi et al., Nature 424: 171-174 (2003); C. M. Niemeyer Trends Biotechnol. 20: 395-401 (2002); 0. G. Schmidt et al., Nature 410: 168 (2001)). Nanomaterials can be used as building blocks for the construction of larger devices and systems, thereby helping to form structures (G. M. Credo et al., J. Amer. Chem. Soc. 124: 9036-9037 (2002); G. L. Baneyx et al., Proc. Natl. Acad. Sci. U.S.A. 99: 5139-5143 (2002); P. Hyman et al., Proc. Natl. Acad. Sci. U.S.A. 99: 8488-8493 (2002); J. Goldberger et al. Nature 422: 599-602 (2003)). Nanoscale devices, due to their small dimensions, are expected to make enormous impacts in biology, chemistry, cancer therapy, computer science and electronics (e.g., 2000, Nanotechnology Research Directions: IWGN Workshop Report; Vision for Nanotechnology R & D in the Next Decade; Eds. M. C. Roco, R. S. Williams and P. Alivisatos, Kluwer Academic Publishers). Nanodevices are currently being commercialized including tissue replacement materials, cancer therapy, multicolor optical coding of biological assays, manipulation of cells and biomolecules, and protein detection. (e.g., O. V. Salata J. Nanobiotechnology 2: 3 (2004)). Nanotechnological endeavors are expected to play critical roles in many scientific disciplines, including chemistry, physics, biology, medicine, materials science, engineering, and computer technology.

Living systems contain a wide variety of nanomachines and other such ordered structures (C. Zandonella Nature 423: 10-12 (2003)) including motors (A. Inoue et al., Nat. Cell Biol. 4: 302-306 (2002); P. Guo Prog. In Nucl. Acid Res. & Mol. Biol. 72: 415-472 (2002); A. Yildiz et al.; Science 300: 2061-2065 (2003); G. Oster et al., Nature 396: 279-282 (2003); R. M. Berry Philos. Trans. R. Soc. Lond. B Biol. 355: 503-509 (2003); D. N. Grigoriev et al., “Bionanomotors” in Nalwa, Ed., Encyclopedia of Nanoscience and Nanotechnology, 1:361-374 (2004); S. D. Moore Curr. Biol. 12: R96-98 (2002); E. P. Sablin et al., Curr. Opin. Struct. Biol. 11: 716-724 (2001)); arrays (W. Shenton et al., Nature 389: 858-587 (1997); J. Carazo et al., J. Mol. Biol. 183: 79-88 (1985); J. Jimenez et al., Science 232: 1113-1115 (1986)), pumps, membrane cores, and valves. The novelty and ingenious design of such machines have helped inspire the development of biomimetrics for nanodevices (C. M. Niemeyer Trends Biotechnol. 20: 395-401 (2002); P. Hyman et al., Proc. Natl. Acad. Sci. U.S.A. 99: 8488-8493 (2002)). Much current research is being devoted to make these machines as viable and effective as possible outside their native environment (E. Dujardin et al., Nano Letters 3(3): 413-417 (2003)). These nanodevices have potential applications in the delivery of drugs (R. K. Soong et al. Science 290: 1555-1558 (2000)) and therapeutic macromolecules (S. Hoeprich et al., Gene Therapy 10(15): 1258-1267 (2003)), the gearing of other nanodevices for purposes such as nanoelectromechanical systems (NEMS) (H. G. Craighead, Science 290: 1532-1536 (2000)), the driving of molecular sorters, the building of intricate arrays and chips for diagnostics, molecular sensors, and novel and complex actuators (A. M. Fennimore et al., Nature 424: 408-410 (2003)) in new electronic and optical devices (H. Hess et al., Reviews in Mol. Biotechn. 82: 67-85 (2001)).

Recently, DNA has been investigated rather extensively for its potential to be used in nanodevices (J. Shi et al., Angew. Chem. 36: 111-113 (1997), N. C. Seeman et al., Proc. Natl. Acad. Sci. U.S.A. 99 Suppl. 2: 6451-6455 (2002), H. Yan et al., Nature 415: 62-65 (2002), H. Yan et al., Proc. Natl. Acad. Sci. U.S.A. 100: 8103-8108 (2003), M. G. Warner et al., Nat. Mater. 2: 272-277 (2003), K. Keren et al., Science 297: 72-75 (2002), D. Gerion et al., J. Amer. Chem. Soc. 124: 7070-7074 (2002)). However, the rigidity of the double-helical structure, and the lack of structure diversity of DNA limits its utility. Stable branched structures with greater structural complexity have been explored by the use of sticky-ends as bridges for linkage between DNA subunits (C. Mao et al., Nature 407: 493-496 (2000), C. J. Nuff et al., Nucleic Acids Res. 30: 2782-2789 (2000), G. A. Soukup et al., Trends Biotechnol. 17: 469-476 (1999)).

Molecular nanomotors are nanostructures that are likely to prove especially valuable as nanotechnology comes of age. The overall significance of nanomotors to nanotechnology is comparable to the impact of the engine in modern society. The ability to harness and utilize, to both construct and deconstruct, these motors has the potential to expand and revolutionize the field of nanotechnology (A. Inoue et al., Nat. Cell Biol. 4: 302-306 (2002), R. K. Soong et al., Science 290: 1555-1558 (2000), G. L. Baneyx et al., Proc. Natl. Acad. Sci. U.S.A. 96: 12518-12523 (1999)).

In living systems, cellular components are actively transported by molecular motors such as F1-ATPase, kinesin, myosin and helicase. During maturation of a DNA virus, the lengthy viral genome is translocated with remarkable velocity by a viral molecular motor into a limited space within a preformed protein shell and packaged to an almost crystalline density. Viral DNA-translocating motors includes both structural (integrated) and nonstructural (transient) components.

Bacterial virus phi29 is an unparalleled system for the study of the mechanism of DNA packaging due to its high efficiency of in vitro DNA packaging (Guo et al., 1986, Proc. Natl. Acad. Sci. USA 83, 3505-3509). The phi29 DNA packaging motor has been reported to be the strongest existing molecular motor with the highest stalling force of 57 pico-newtons and a speed of 100 bases per second (Smith et al., 2001, Nature 413, 748-752). The viral motor performs the DNA packaging reaction. Neck protein gp11/12, tail protein gp9, and morphogenic factor gp13 are needed to complete the assembly of infectious virions. The structure of connector protein gp10 has been solved by X-ray crystallography (Simpson et al., 2000, Nature 408, 745-750; Guasch et al., 2002, J. Mol. Biol. 315, 663-676). The pRNA has been shown to form a hexamer to gear the DNA-packaging motor (Guo et al., 1998, Mol. Cell. 2, 149-155; Trottier and Guo, 1997, J. Virology, 71,487-494; Hendrix, 1998, Cell 94, 147-150; Zhang et al., 1998, Mol. Cell. 2, 141-147).

All components needed to package phi29 DNA and to assemble infectious virions have been purified and can be used for in vitro assembly of the motor. The in vitro assembly system can convert a DNA-filled capsid into an infectious virion. With this efficient system, up to 10⁸ pfu/ml of infectious virions can be assembled in vitro, while the omission of a single component results in no plaque formation (Lee et al., 1994, Virol, 202, 1039-1042; Lee et al., 1995, J. Virol. 69, 5018-5023).

The operation of a motor requires energy. In addition, to ensure the continuous motion of the motor, at least one component should act processively. In living organisms, the intriguing process of bioenergy conversion is manifest in ATP binding and hydrolysis. All bio-motors such as myosin, kinesin, DNA-helicase and RNA polymerase involve an ATP-binding component that acts processively.

ATPase activity has been long believed to be possessed by proteins only. It is generally believed, for example, that gp16 is the processive factor in driving the phi29 DNA-packaging motor. However, RNA is much easier to synthesize than proteins, and a molecular motor powered by an RNA that participates in the generation of ATPase activity would find broad use in medical and nanotechnology applications.

### SUMMARY OF THE INVENTION

The invention provides a molecular motor, termed herein a “molecular nanomotor” or simply “nanomotor,” capable of translocation of a polynucleotide. The molecular nanomotor of the invention comprises a nanoscale structure formed from the association of both protein and RNA. In one embodiment, the nanomotor is derived from a phi29 bacteriophage nanomotor and contains structural components that include a connector protein gp10, a capsid protein gp8, and a pRNA, or their equivalents. These structural components together form a nanoscale structure capable of effecting translocation of a polynucleotide in the presence of a gp16 protein, ATP and Mg⁺⁺. Optionally, protein gp7 can be included in the nanomotor as a structural component.

Two other components of the nanomotor, a gp16 protein and ATP, are considered “nonstructural.” Although they are not structurally integrated into the nanomotor, these components impart functionality to the nanomotor. These nonstructural components are transiently associated with the structural part (i.e., the nanoscale structure) of the nanomotor. In order for the nanomotor to function, the nanomotor should be supplied with gp16, ATP and magnesium (Mg⁺⁺). An optional nonstructural component which is expected to enhance the function of the nanomotor is polyethyleneglycol (PEG), which enhances the solubility of gp16. The solubility of gp16 can likewise be enhanced by adding selected amino acids to the N-terminus that, for example, increase the hydrophilicity of gp16 and/or inhibit nonspecific aggregation.

Translocation activity of the nanomotor can be reversibly halted by contacting the nanomotor with a chelating agent, contacting the nanomotor with a nonhydrolyzable ATP analogue, or depriving the nanomotor of a source of gp16 protein, ATP and/or Mg⁺⁺. Activity resumes when the nanomotor is supplied with additional Mg⁺⁺, ATP, or gp16 protein, depending on the method used to reversibly stop the nanomotor. Translocation activity of the nanomotor can be irreversibly stopped by contacting the nanomotor with RNase, which degrades the pRNA component.

The invention provides a method for translocating a polynucleotide that involves providing a molecular nanomotor having a nanoscale structure according to the invention, and contacting the nanoscale structure with a gp16 protein, ATP, Mg⁺⁺ and, optionally, PEG, under conditions effective to translocate the polynucleotide. The polynucleotide that is translocated can be linked, covalently or noncovalently, to a molecular cargo that is also translocated. Optionally, the method includes reversibly stopping the nanomotor, for example by contacting the nanoscale structure with a metal chelating agent such as EDTA or a nonhydrolyzable ATP analogue such as γ-S-ATP. The nanomotor can then be restarted as described above. The nanomotor may be irreversibly stopped by contacting it with RNase.

The nanomotor of the invention exhibits many important and unusual characteristics. For example, the nanomotor is a rotational (rotary) motor (FIG. 18). RNA serves together with proteins as a motor component, resulting in a composite RNA-protein motor structure. The rotary nanomotor contains a 6 “pole” rotor element formed from pRNAs, the connector, and gp16 protein, and a 5-“pole” stator element made by the procapsid. The rotation of the nanomotor is counterclockwise when viewed from the portal side, suggesting that DNA packaging is achieved by utilizing the “threaded” helical nature of dsDNA. The “differential” effect of this rotary motor is due to the symmetry mismatch between the “rotor” and the “stator” of the packaging motor.

Significantly, in this unique motor the RNA component binds ATP and is part thus of the ATPase activity, thereby being involved in providing fuel to the motor. Synthetic pRNA as well as naturally occurring pRNA can be utilized, as described in more detail below. Surprisingly, the ATP-binding RNA (whether naturally occurring or synthetic, as described more fully below) has the ability to drive the nanomotor. The pRNA can be manipulated and controlled at will to form dimers, trimers and other structures with different shapes and sizes (FIG. 19) and can be derivatized with groups for linking to other components during the construction of the macromolecular complex. To that end the pRNA optionally includes a 3′ pRNA extension region. The 3′ extension region can include a capture region, for example a capture region that hybridizes to a polynucleotide, and/or a reactive group, e.g., for attachment of the molecular nanomotor to a substrate.

Advantageously, the molecular nanomotor of the invention as well as the pRNA molecules of the invention can serve as building blocks in nanotechnology. One example is the use of the molecular nanomotor of the invention as a device for sorting polynucleotides. The invention provides a method for sorting biomolecules, particularly polynucleotides, making use of a molecular nanomotor that includes, as a pRNA component, a pRNA having a 3′ extension region having a capture region that selectively hybridizes to a polynucleotide. The method involves contacting the molecular sorting device with a mixture of polynucleotides under conditions that permit selective hybridization of the polynucleotide to the 3′ extension region followed by translocation of the selected polynucleotide.

In another aspect, the invention provides microarray formed from a multiplicity of pRNA molecules, which pRNA molecules can be the same or different. Such a microarray can function, for example, as a lattice or scaffolding. The pRNA molecules used to form the microarray can be naturally occurring or non-naturally occurring. The microarray can include any desired pRNA structure, such as a pRNA monomer, dimer, trimer, tetramer, hexamer, twin or double twin. The array can be extended using interactions between intramolecularly and/or intermolecularly complementary nucleotide sequences present on the right and/or left loops of the pRNA constituents. Other forms of pRNA that can be used in the microarray include pRNA molecules that have palindromic 3′ and 5′ ends, and pRNA molecules that are circularly permuted (cpRNA).

In embodiments containing pRNA monomers, preferably at least a portion of the pRNA monomers include a helical junction region resulting in an odd number of half-turns. The odd number of half turns extends the area between the two monomers to allow for continued array growth.

In another preferred embodiment of the microarray, at least a portion of the pRNA molecules form a shape selected from a checkmark, a rod, a triangle, a bundle, a spiral and a hairpin.

The pRNA used in the microarray can be shorter (truncated) or longer (extended) than wild-type pRNA. If shorter (truncated), the pRNA preferably includes a region that has the same three-dimensional structure as bases 23 through 97 of phi29 pRNA. If longer, the pRNA preferably includes an extension region on the 3′ end. The extension region optionally contains a capture region, for example to allow a polynucleotide to hybridize to the pRNA, for example to facilitate translocation of the polynucleotide. Additionally or alternatively, the 3′ extension region may include a functional group such as a reactive group for attachment to a substrate.

The microarray of the invention can be a two-dimensional or three-dimensional array. It can be attached to a substrate (immobilized) or present in solution.

The invention is further directed to a nanoscale device that includes a molecular nanomotor or component thereof, a microarray, or a pRNA of the invention.

### BRIEF DESCRIPTION OF THE DRAWINGS

FIG. 1 is a graphical representation of the phi29 DNA packaging motor. Panels A and B show the 3D structure of the nanomotor with bottom view and side view, respectively. Panel C is a space filling model of the structure of aptRNA predicted by computer modeling based on experimental data derived from photo-affinity cross-linking, chemical modification and chemical modification interference, complementary modification, nuclease probing, and cryo-AFM.

FIG. 2 shows the use of the poorly hydrolysable ATP analogue γ-S-ATP to halt the motor and produce DNA-packaging intermediates. It also shows that the halted motor can be restored to function, since the intermediates can be converted into infectious virus after the addition of ATP.

FIG. 3 is a graph demonstrating the requirement of pRNA and gp16 for the initiation of DNA packaging by conversion of phi29 DNA-packaging intermediates into infectious virion. “Omit gp16” or “omit pRNA” indicates that during the first DNA packaging step, either gp16 or pRNA, respectively, was omitted from the DNA packaging mixture. “Complete” indicates the complete insertion of the entire genomic DNA into the protein shell. The incomplete DNA-packaging intermediates in each fraction of the gradient were subsequently converted into infectious phi29 virion by the addition of fresh gp16, ATP, neck protein gp11/12, and tail protein gp9.

FIG. 4 shows graphs demonstrating the requirement of fresh gp16 and ATP but no requirement of pRNA for the motor to continue and complete the DNA packaging of intermediates. Each fraction of the gradient containing DNA-packaging intermediates were subsequently converted into infectious phi29 virion in the absence of (a) pRNA; (b) gp16; (c) ATP; or (d) in the presence of RNase to cleave the pRNA in the intermediates.

FIG. 5 shows a schematic representation of the structure of phi29 DNA packaging nanomotor (Hoeprich and Guo, J. Biol Chem, 277, 20794-20803, 2002). A. Computer model of three-dimensional structure of phi29 pRNA monomer based on experimental data derived from photo-affinity cross-linking; chemical modification and chemical modification interference; complementary modification; nuclease probing; and cryo-AFM. B. pRNA hexamer docking with the connector crystal structure that has a 3.6 nm central channel for DNA entry during packaging (Simpson et al., 2000, Nature 408, 745-750). Six pRNA molecules are linked by hand-in-hand interaction via the right hand loop and left hand loop.

FIG. 6 shows ATP-binding assay with ATP-agarose affinity column. A: Binding of pRNA_{wt} (A-I) and aptRNA (A-II) to ATP (Shu and Guo, J. Biol Chem, 278, 7119-7225, 2003). B. Binding of pRNA_{wt} to ATP-affinity column and elution with ADP or GTP (B-I), as well as UTP or CTP (B-II). Each insert in B shows the entire spectrum of the elution profile.

FIG. 7 shows a comparison of the central region of pRNA with the ATP-binding RNA aptamer. A. Sequence comparison of the (a) central region (SEQ ID NO:1) of pRNA_{wt} (SEQ ID NO:2) (Guo et al., 1987, Nucleic Acids Res. 15, 7081-7090; Bailey et al., 1990, J. Biol. Chem. 265, 22365-22370) with (b) the 40-base ATP-binding RNA aptamer, ATP-40-1 (SEQ ID NO:3) (Sassanfar et al., 1993, Nature 364, 550-553; Cech et al., 1996, RNA 2, 625-627). The similar bases are in lower case letters. G^{con} is a conserved base essential for ATP-binding (Shu and Guo, J. Biol Chem, 278, 7119-7225, 2003). B. Structure comparison of (a) the central region of pRNA with (b) the ATP-binding RNA aptamer. The 3D structures in c and d are derived from computer modeling and NMR (Dieckmann et al., 1996, RNA 2, 628-640), respectively. The 5′ and 3′-ends of the moiety in c and d are marked. The adenosine residue is marked (Dieckmann et al., 1996, RNA 2, 628-640). C. Sequence of the chimeric aptRNA and related mutant aptG^{con}C (SEQ ID NO:4) (see PCT WO 02/16596). D. Comparison of concentration requirement between chimeric aptRNA and wild type pRNA_{wt} in phi29 assembly E. Sequences of pRNAs from (a) SF5, (SEQ ID NO: 5), (b) BIO3, (SEQ ID NO: 6), (c) phi29/PZA, (SEQ ID No: 2), (d) M2/NF, (SEQ ID NO: 7), and (e) GA1, (SEQ ID NO: 8); the 3′ ends have been extended to facilitate recombinant expression.

FIG. 8 shows in vitro production of infectious virions of phi29 particles with aptRNA and ATP (Shu and Guo, J. Biol Chem, 278, 7119-7225, 2003). A. Electron microscopy (EM) image (×90,000) of purified phi29 procapsid devoid of genomic DNA. B. Plaques formed on a lawn of Bacillus subtilis after plating with the infectious virus produced from the reaction with aptRNA and ATP. C. EM image (×90,000) of the viral particles purified from the lawn in B. D. Agarose gel showing EcoRI restriction mapping of genomic DNA from wild-type phi29 (lane b), and from the virus assembled with aptRNA (lane c). Lane d shows 1-kb ladder and lane a contains a control sample from procapsid (A) that is devoid of genomic DNA.

FIG. 9 shows ATP binding affinity of pRNA and aptRNA (Shu and Guo, J. Biol Chem, 278, 7119-7225, 2003). A-B. [³H]aptRNA (A) or [³H]pRNA_{wt} (B) was applied to a column (0.55 cm in diameter) packed with ATP-C-8 affinity agarose (0.8 ml) and eluted with a 2 ml step-up gradient with specified concentration of ATP in binding buffer. Fractions were collected and subjected to scintillation counting. C. [³H]aptRNA was applied onto a 0.8 ml ATP-agarose affinity column and washed with binding buffer, then eluted with buffer containing 0.004 mM of ADP (C-I), UTP (C-II), CTP (C-II) or GTP (C-II), then with 0.004 mM ATP. Arrows indicate that the given concentration of specified nucleotides was added to the binding buffer. Each fraction is 250 μl.

FIG. 10 shows sequences of wild type and mutant pRNAs used in confirmation verification studies (Shu and Guo, J. Biol Chem, 278, 7119-7225, 2003). The left panel (A-D) (SEQ ID NOs: 2, 9, 10 and 11, respectively) is a set of deletion mutants derived from the wild type parental pRNA_{wt} to confirm the conformation of mutants with a change of G^{con} (in A, B and C) to C (in D). The right panel (E, F, G and H) (SEQ ID NOs: 4, 4, 12 and 13, respectively) is a set of deletion mutants derived from parental aptRNA to confirm the conformation of mutant with a change of G^{con} (E and G) to C (F and H). The plot of (I) shows a competitive inhibition assay to compare the conformation of pRNA with and without the mutation of G^{con}.

FIG. 11 is a native gel electropherogram depicting the interaction of ATP-binding RNA with ATP. Lane a, 5S rRNA, no ATP; lanes b-c, 5S rRNA, increasing amounts of ATP; lane d, DNA ladder; lane e, aptRNA, no ATP; lanes f-h, aptRNA, increasing amounts of ATP (Shu and Guo, J. Biol Chem, 278, 7119-7225, 2003).

FIG. 12 is an autoradiogram of an ATPase assay by thin layer chromatography showing the hydrolysis of [γ-³²P]ATP in the presence of pRNA (Shu and Guo, J. Biol Chem, 278, 7119-7225, 2003).

FIG. 13 depicts the results ATP-binding assay with ATP-agarose affinity column. A. Binding of aptRNA (◯); aptGconC (▪); and 116-base rRNA control (▴) to ATP-agarose affinity column. B. Elution of aptRNA from the column using ADP (◯) and ATP (▴). C. Elution of aptRNA using UTP (♦); CTP (▪); GTP (◯); and ATP (▴) (Shu and Guo, J. Biol Chem, 278, 7119-7225, 2003).

FIG. 14 depicts reversibility of motor function. The motor shut off by γ-S-ATP could be turned-on again by ATP. ATP, gp16, gp11/12 and gp9 were added to each fraction from the sucrose gradient containing DNA-packaging intermediates, which were blocked by γ-S-ATP, and assayed for the production of infectious virus. When ATP was added (◯), viruses were produced. However, in the absence of ATP (▪), or when EDTA (▴) or RNase (X) were added, no viruses were produced, indicating that EDTA and RNase blocked the motor.

FIG. 15 depicts the passive release of DNA from protein complex. A. At pH 4 (lane c), but not pH 7 (lane b), phi29 DNA was released from the protein shell and was sensitive to EcoRI digestion, similar to purified phi29 DNA (lane d). B. Electron micrograph shows the released DNA (arrow), with TMV virus (a bar at the top) as size control.

FIG. 16 depicts binding experiments used to determine the apparent dissociation constant K_{D,app} for RNA/ATP interaction. A. Isocratic elution for ATP that was immobilized on agarose (ATP_{bound}); B. ATP gradient elution for free ATP (ATP_{free}) (Shu and Guo, J. Biol Chem, 278, 7119-7225, 2003).

FIG. 17 depicts the sequential action of pRNAs in a phi29 DNA packaging motor. The leftmost image is the three-dimensional structure of the motor complex including the connector and pRNA hexamer. In A-G showing the six steps of rotation, the hexagon represents the phi29 connector and the surrounding pentagon represents the capsid. Six protrusions represent six pRNAs with variable pRNA patterns portraying the pRNA in serial energetic states. For example, pRNA 4 and 1 in panel A represent contracted and relaxed conformations, respectively. Arrows mark the different transition states of pRNA 1. Each step, e.g., A to B, rotates 12°, since a five to six-fold symmetry mismatch generates 30 equivalent positions, and 360°/30=12°. The portal vertex turns 72° after six steps of rotation. For example, pRNA 1 moves from vertex a in A to vertex b in G, and rotates 72°. Each step consumes one ATP to induce one conformation change of pRNA, and six ATPs are used for the transition from one vertex to another.

FIG. 18 illustrates the formation of pRNA dimers and trimers with variable shapes. Dimers and trimers that have all of the left and right hand loops bound via hand-in-hand interactions are called “closed”. Dimers and trimers that have one left and one right hand loops not bound via hand-in-hand interactions are called “open”. Open dimers and trimers are shown with an “X” between the left and right hand loops that do not base pair. Cartoons illustrate the concept of hand-in-hand interactions. The right column is the direct observation of purified monomer, dimer and trimer with Cryo-AFM (Atomic Force Microscope). RNA monomers, dimers and trimers with variable lengths are likely to be useful in the construction of nanodevices.

FIG. 19 illustrates the potential application of the DNA-packaging motor as a molecular sorter. Specific sequences can be added to the 3′ end of each of the six pRNA without compromising functionality. The specific recognition of the substrate molecule by the special motor pRNA will aid in identifying and picking up given molecules from within the mixed population.

FIG. 20 shows the sequences of full-length (120 base), truncated (e.g., 23/97) and extended pRNAs used in Example III.

FIG. 21 shows the secondary structure of a trimer made of a normal (SEQ ID NO:25), truncated (SEQ ID NO:24), and extended (SEQ ID NO:25) pRNA. The truncated pRNA is at the top (B-e′), the normal pRNA is on the right (A-b′), and the extended pRNA is on the left (E-a′). The upper case letters describe the right loop of the pRNA and the lower case letters describe the left loop.

FIG. 22 illustrates open and closed dimers and trimers. The two types of pRNA shown are the 5′/3′ pRNA and the circularly permutated pRNA (cpRNA) (K. Garver et al., J. Biol. Chem. 275(4): 2817 (2000)). Dimers and trimers that have all of the left-hand and right-hand loops bound via hand-in-hand interactions are called “closed.” Dimers and trimers that have one left-hand loop and one right-hand loop not bound via hand-in-hand interactions are called “open.” Open dimers and trimers are shown with an X between the left-hand and right-hand loops that do not base pair. Cartoons illustrate the concept of hand-in-hand interactions. The right column is the direct observation of purified monomer, dimer, and trimer with a cryo-atomic force microscope.

FIG. 23 illustrates an assortment of dimers and trimers with full-length and truncated pRNAs.

FIG. 24 is an audioradiogram of an 8% native polyacrylamide gel showing different monomers, dimers, and trimers before purification.

FIG. 25 shows (A) a graph showing the isolation and separation of stable multimers: 5-20% sucrose gradient sedimentation to separate [³H]-dimer and trimer isolated from native polyacrylamide gel. (A-b′)/(B-a′) dimer centering at fraction 8 runs faster than (A-b′) monomer itself centering at fraction 12. The (A-b′)/(B-e′)/(E-a′) trimer ran faster (peaked at fraction 6) than the dimer. Sedimentation is from right to left. (B) a plot of hypothetical molecular weight vs. the log of migration distance (the fractional number) in gradient.

FIG. 26 is a representation of the computer modeling of (a) the 3D structures of pRNA dimers (S. Hoeprich et al., J. Biol. Chem. 277(23): 20794 (2002)) and illustration of the phi29 procapsid from (b) side and (c) bottom views.

FIG. 27 shows graphs demonstrating the inhibition of phi29 viral assembly by assorted inactive dimers and trimers. Different amounts of assorted competitive dimers or trimers were mixed with a constant amount of wild-type pRNA before being applied in in vitro assembly assays. Inhibition of phi29 assembly by assorted dimers or trimers suggests that the assorted dimers and trimers, though inactive, contain an unchanged conformation for procapsid binding.

FIG. 28 shows graphs demonstrating (A) the sucrose gradient showing the effects of ions on dimerization. Equal molar ratios of the [³H] A-b′ and cold B-a′ were mixed together and loaded on top of the gradients containing different ions. (B) a plot of hypothetical molecular weight vs. the log of migration distance (the fractional number) in gradient.

FIG. 29 shows a table of the conditions affecting pRNA oligomerization and the stability of oligomers after complex formation.

FIG. 30 shows an SDS-PAGE gel stained with Commassie Blue illustrating the test of the stability of pRNA dimers under different conditions. The slower migration in lane 6 is due to the high salt concentration's effect during electrophoresis. RNase A, however, did affect the formation of dimers by digesting the monomer subunits. The absence of a monomer band in lanes 17-20 indicates that RNase A did not simply interfere with the hand-in-hand interactions. For lanes 7-8, dimer RNAs were exposed to different pH buffers before native gel. In lanes 10-15, dimers RNAs were incubated at different temperatures for 10 minutes before being applied to native gel.

FIG. 31 illustrates the sequence and structural elucidation of phi29 motor pRNA and related assemblages: (A) the primary and secondary structure of wild-type pRNA I-i′. The binding domain (shaded area) and the DNA translocation domain (the helical region) are marked with bold lines. The four bases in the right and left loops, which are responsible for inter-RNA interactions, are boxed; (B) the three-dimensional structure of wild-type pRNA I-i′ displayed as ribbon (S. Hoeprich et al., J. Biol. Chem. 277(23): 20794-20803 (2002)); (C) diagrams depicting the pRNA monomer A-b′ with unpaired right/left loops; (D) pRNA dimers (A-b′)(B-a′); (E) pRNA trimers (A-b′)(B-e′)(E-a′); (F) pRNA monomer with unpaired right/left loops A-b′ and a 6-nucleotide palindromic sequence; (G) pRNA twin A-b′.

FIG. 32 shows SDS-PAGE gel stained with Commassie Blue showing monomers, dimers, trimers, twins, tetramers, and arrays: (A) native and denatured gel; (B) test of the stability of pRNA dimers under different conditions.

FIG. 33 is a graph illustrating the separation of pRNA monomers, dimers, trimers, twins and arrays by 5-20% sucrose gradient sedimentation. The [³H]-pRNA monomers, dimers, trimers and twins were isolated from native polyacrylamide gel (see Example IV). Arrays were prepared by mixing of equal molar amount of twin (A-b′), twin (B-e′) and twin (E-a′). All particles were loaded onto the top of the gradient and sedimented by ultracentrifugation. Sedimentation is from right to left.

FIG. 34 illustrates the Atomic Force Microscopy (AFM) showing pRNA monomers (A), dimers (B), trimer (C) and arrays (D) of pRNA. The three inserts at the left of each panel contain images with higher magnification, as indicated by the size of the frame. The pRNA monomers folded into a checkmark shape, dimers displayed a rod shape, trimer exhibited triangle shape, and arrays displayed as bundles. Formation of dimers requires Mg⁺⁺, while the sample on mica was briefly rinsed with water before freezing for cryo-AFM, which resulted in some dissociation of dimers or trimers even when the pRNA was already adsorbed to the activated mica surface. The contrast within each image reflects the thickness and height of the molecule. The brighter, or whiter the image, the thicker or taller the molecule; the darker the image, the thinner the molecule.

FIG. 35 illustrates a mixture of two complementary twins, A-b′ and B-a′, assembled into two distinct supramolecular structures. (A) Two complementary twins were able to form a stable tetramer (double-twins) by assembling into a circular structure. (B) Concatemers of alternating twins formed when a twin interacted with two rather than one complementary twin.

### DETAILED DESCRIPTION OF ILLUSTRATIVE EMBODIMENTS

The construction of nanoscale artificial motors by chemical synthesis is an intriguing endeavor in contemporary technology. We show here that a 30-nanometer motor can be made in vitro with purified recombinant proteins and artificially designed RNAs.

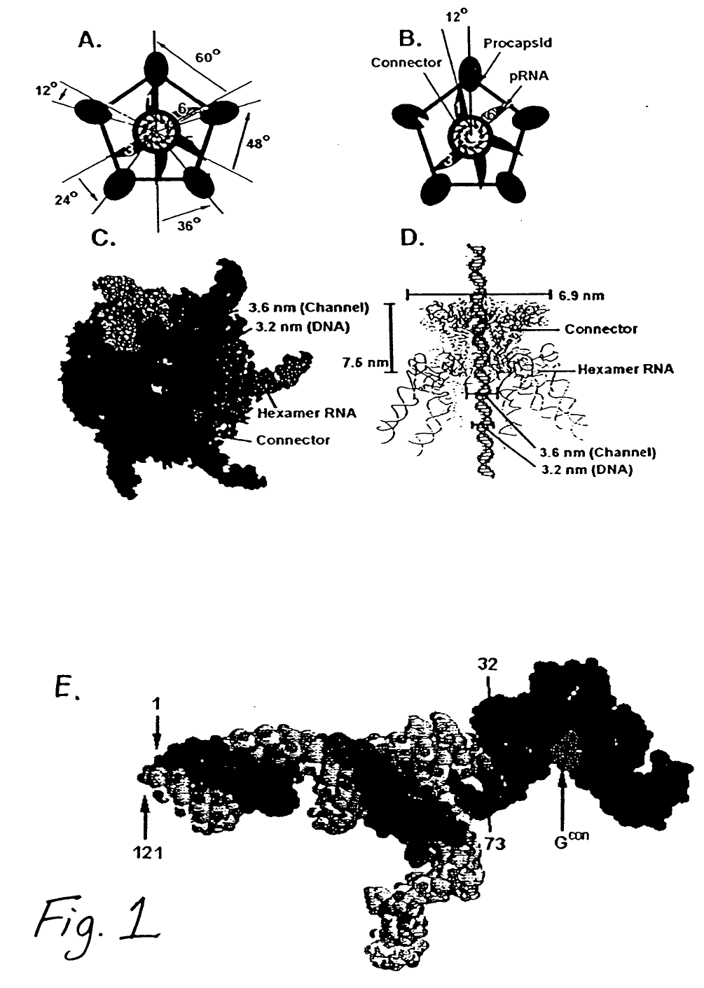

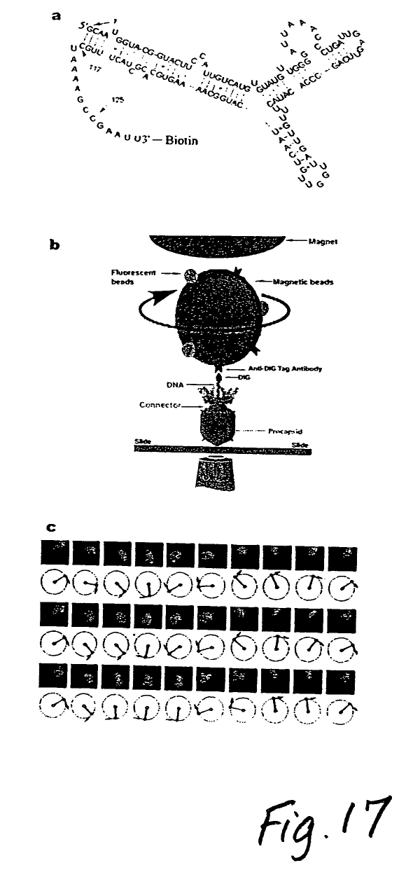

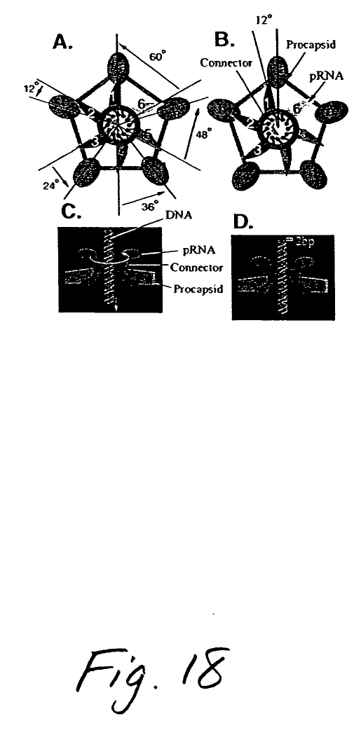

The 30-nanomotor exemplified herein is modeled on the sequential action of pRNA in phi29 DNA packaging (FIGS. 1, 17, and 18). Phi29 contains a capsid with a five-fold symmetry. Interaction of the hexamer RNA and the capsid generates a five to six-fold symmetrical match to facilitate a continuous rotation of the motor. Each step rotates 12°, since a five to six-fold mismatch generates 30 equivalent orientations (360°/30=12°). The variable shapes and patterns portray six RNA in serial energetic states. Each 12° rotation will move one of six RNA to align with one vertex of the pentagon. The portal vertex turns 72° after six steps. For example, RNA #2 moves 12° from panel A to touching the vertex b in panel B. In A, RNA #1 is aligned with vertex a, while in B, RNA #1 is 12° away from vertex a. Each 12° increment consumes one ATP. Therefore, 30 ATPs are needed for one 360° rotation.

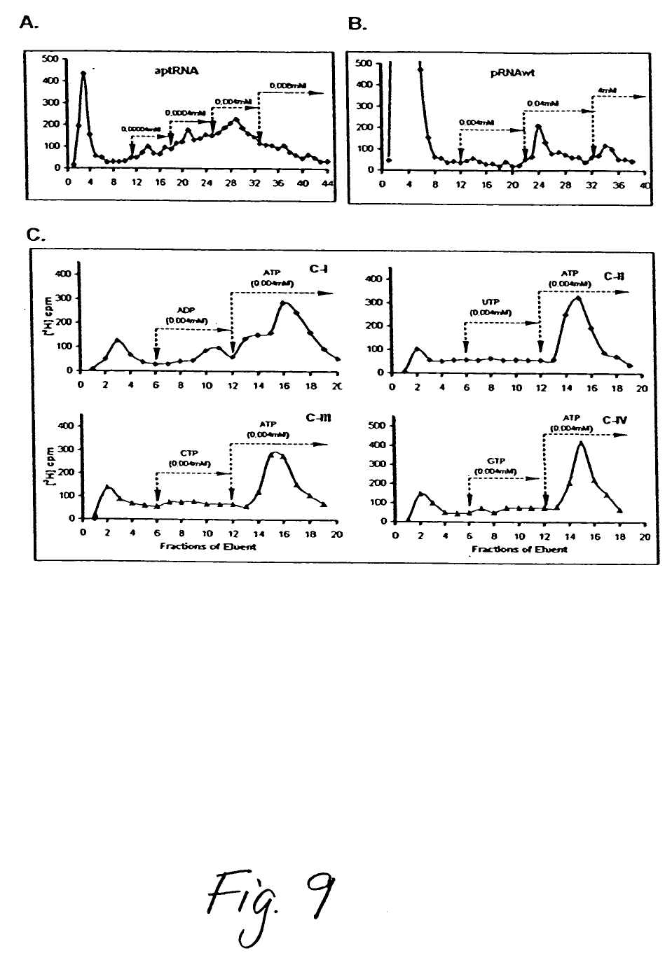

ATP-binding RNA, dubbed aptamer, was identified from synthesized random RNA pools using a chemical in vitro selection and amplification technique. A 40-base RNA aptamer was selected chemically and found to be able to bind ATP. Using this 40-base ATP-binding RNA aptamer as a central element (FIG. 7A(a)), a chimeric 121-base pRNA, called aptRNA, was constructed (FIG. 7C) to imitate the DNA-packaging pRNA of bacterial virus phi29. Amazingly, this aptRNA was able to power the protein complex to pump the viral DNA genome into the protein shell, and to produce infectious virus in the test tube. ATP is used as the source of energy. The mechanics of the motor resemble the driving of a bolt with a hex nut with six pRNAs forming a hexagonal complex to gear the DNA translocating machine in 12° increments.

Importantly, the processive factor in the phi29 DNA-packaging motor was discovered to be the pRNA not gp16. The pRNA is a structural part of the nanomotor and also acts as an enzyme, constantly working. The protein gp16, on the other hand, appears to be transiently associated with the complex, although it is, nonetheless, apparently required for the first round of assembly, and needs replenishment if the motor is to function; it is a transient distributive factor in motor function. For the nanomotor to function, a continuous supply of gp16, ATP and Mg⁺⁺ is needed.

Protein gp16 optionally contains an extension on the N-terminus. The N-terminal extension region may include one or more amino acids and/or functional groups other than, and in addition to, amino acids (e.g., a biotin molecule). An N-terminal extension can, for example, increase the solubility of gp16 and/or facilitate its purification. The solubility of gp16 can be enhanced, for example, by adding selected amino acids to the N-terminus that, for example, increase the hydrophilicity of gp16 and/or inhibit nonspecific aggregation. The addition of an N-terminal extension region may also increase the activity of gp16. Purification of gp16 can be enhanced, for example, by including a “histidine tag” (a series of histidine residues) that facilitate affinity purification. The extension region may also, for example, include a binding site for facilitating association of a polynucleotide with the nanomotor prior to translocation of the polynucleotide, a reactive group for attachment or tethering of the nanomotor to a substrate, and/or a detectable label for identifying or tracking the molecular motor.

The molecular nanomotor can be reversibly turned off by the addition of a nonhydrolyzable ATP analog, e.g., γ-S-ATP or a metal chelating agent, such as EDTA. If a nonhydrolyzable ATP analog is used to turn off the nanomotor, it can be restarted by adding ATP. If EDTA or other chelating agent is used to turn off the nanomotor, the addition of magnesium will restart it. The nanomotor can also be reversibly turned off by depriving the nanomotor of the distributive factor, gp16, or depriving it of ATP, thereby eliminating the fuel source. The nanomotor can be restarted with the addition of fresh gp16 or ATP, respectively. Irreversible shut-down of the nanomotor can be accomplished by treating the nanomotor with RNase, which compromises its structural integrity by degrading the pRNA component.

Component Proteins

The proteins described herein for use as components of the molecular nanomotor can include naturally occurring or synthetic sequences. In other words, although a preferred embodiment of the nanomotor utilizes protein components in their naturally occurring form, proteins that are structurally and functionally equivalent can be used. Unless otherwise indicated herein, when a structural or nonstructural protein component of the nanomotor, such as “protein gp16” is referred to herein, that term includes proteins that are both structurally and functionally equivalent to the protein referred to. The proteins used as components of the nanomotor can be isolated directly from bacteriophage, produced recombinantly, or enzymatically or chemically synthesized.

Structural equivalency can be defined by reference to the level of amino acid identity between the sequence of the candidate protein used in the nanomotor and the corresponding reference, naturally occurring sequence. Preferably, a structurally equivalent protein has an amino acid sequence that shares at least an 80% amino acid identity to the corresponding naturally occurring sequence. Amino acid identity is defined in the context of a homology comparison between the candidate sequence and the reference sequence. The two amino acid sequences are aligned in a way that maximizes the number of amino acids that they have in common along the lengths of their sequences; gaps in either or both sequences are permitted in making the alignment in order to maximize the number of shared amino acids, although the amino acids in each sequence must nonetheless remain in their proper order. The percentage amino acid identity is the higher of the following two numbers: (a) the number of amino acids that the two polypeptides have in common within the alignment, divided by the number of amino acids in the candidate protein, multiplied by 100; or (b) the number of amino acids that the two polypeptides have in common within the alignment, divided by the number of amino acids in the reference protein, multiplied by 100. It should be understood that structural equivalents of a protein can included derivatives of a protein (e.g., proteins that have been altered by amidation, acetylation and the like) as well as proteins having deletions or additions with respect to the reference protein (e.g., truncated proteins).

Functional equivalency of a candidate protein is defined as retention of at least a portion of the reference protein's binding or enzymatic activity. Structural proteinaceous components of the nanomotor should retain an ability to associate with (bind) other structural components of the nanomotor. Nonstructural proteinaceous components of the nanomotor should retain an ability to transiently associate with the nanomotor structure and should exhibit at least a portion of the protein's enzymatic activity (e.g., in the case of gp16, the ability to perform the distributive function). The binding and/or enzymatic activity of the various proteins used as components in the nanomotor described herein can be readily determined by evaluating the efficacy of DNA packaging and/or viral assembly assay as set forth in detail in the Examples below.

One of skill in the art of protein biochemistry will appreciate that there are a number of conservative changes that can be made to the amino acid sequence of the reference protein without significantly altering its binding characteristics or other activity. These changes are termed “conservative” mutations, that is, an amino acid belonging to a grouping of amino acids having a particular size or characteristic can be substituted for another amino acid, particularly in regions of the protein that are not associated with catalytic activity or binding activity, for example. Substitutes for an amino acid sequence may be selected from other members of the class to which the amino acid belongs. For example, the nonpolar (hydrophobic) amino acids include alanine, leucine, isoleucine, valine, proline, phenylalanine, tryptophan, and tyrosine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Particularly preferred conservative substitutions include, but are not limited to, Lys for Arg and vice versa to maintain a positive charge; Glu for Asp and vice versa to maintain a negative charge; Ser for Thr so that a free —OH is maintained; and Gln for Asn to maintain a free NH₂.

Component pRNA

The nanomotor requires, as a structural component, a pRNA molecule that binds ATP. The pRNA molecule contains a central ATP binding region, flanked by binding regions that facilitate association of the RNA with the other structural components to form the nanomotor structure. In a preferred embodiment, the flanking regions contain ribonucleotides 1-32 and 69-117 of naturally occurring phi29 RNA (FIG. 7). However, the specific sequence of pRNA is not critical; the important feature of the pRNA is that the secondary and tertiary (3D) structures are similar to native pRNA, allowing the pRNA to bind phi29 procapsid.

The central region involved in ATP binding comprises ribonucleotides 33-68, and it has been found that these nucleotides can be substituted with another ATP binding sequence without affecting motor function (Shu and Guo, J. Biol Chem, 278, 7119-7225, 2003).

Additional ribonucleotides, whether or not derived from naturally occurring phi29 pRNA, can be attached to the 5′ and 3′ends of the pRNA. As noted above, it has been found that up to about 120 ribonucleotides, and maybe more, can be attached to the 3′ end of the pRNA without affecting pRNA folding and function.

The pRNA component of the nanomotor can include naturally occurring or synthetic ribonucleotide sequences. It has been surprisingly found that non-naturally occurring pRNA (e.g., a chimeric pRNA containing aptRNA, as described below, and pRNAs described in Chen et al. (1999, RNA 5, 805-818), Zhang et al. (1994, Virol. 201, 77-85) and Zhang et al. (1997, RNA 3, 315-322) and FIG. 7, can function in the nanomotor. Thus, pRNAs that are structurally and functionally equivalent to native bacteriophage phi29 pRNA can be used in the nanomotor. Unless otherwise indicated herein, when pRNA is referred to herein as a structural component of the nanomotor, that term includes RNAs that are structurally and functionally equivalent to phi29 pRNA.

Structural equivalency can be defined by reference to the level of ribonucleotide identity between the sequence of the candidate pRNA used in the nanomotor and a reference pRNA sequence, such as that derived from bacteriophage phi29. The regions that flank the central, ATP binding region of the candidate pRNA are preferably at least 60% identical to, more preferably 80% identical to, even more preferably 90% identical to, and most preferably 95% identical to the corresponding ribonucleotide sequence of native phi29 pRNA or the pRNA sequence of pRNA (SEQ ID NO: 2) sequences derived from phage SF5 (SEQ ID NO: 5), B103 (SEQ ID NO: 6), M2/NF (SEQ ID NO: 7) or GA1 (SEQ ID NO: 8) which exhibit the same secondary tertiary structure as phi29 pRNA (see FIG. 7). Percent identity is determined by aligning two polynucleotides to optimize the number of identical nucleotides along the lengths of their sequences; gaps in either or both sequences are permitted in making the alignment in order to optimize the number of shared nucleotides, although the nucleotides in each sequence must nonetheless remain in their proper order. For example, the two nucleotide sequences are readily compared using the Blastn program of the BLAST 2 search algorithm, as described by Tatusova et al. (FEMS Microbiol Lett 1999, 174:247-250). Preferably, the default values for all BLAST 2 search parameters are used, including reward for match=1, penalty for mismatch=−2, open gap penalty=5, extension gap penalty=2, gap x_dropoff=50, expect=10, wordsize=11, and filter on. In addition or alternatively, the pRNA used in the nanomotor contains at least 8, more preferably at least 15, most preferably at least 30 consecutive ribonucleotides found in native phi29 pRNA. In the central region of the pRNA, structural equivalence to phi29 pRNA is desirable but not required.

Functional equivalency of a candidate pRNA is defined as retention of at least a portion of the ability to bind ATP, and to associate with the structural proteinaceous components of the nanomotor to form a nanomotor structure with ATPase activity. ATP binding activity is preferably found in the central region of the pRNA. In the motor, gp16 together with pRNA form a functional hexameric ATPase. It should be noted that, for ATP binding activity to be retained, nucleotide G^{con} (FIG. 7) should be retained.

Nanomotor Applications

The nanomotor's basic function of translocating a polynucleotide from one location to another gives it utility in a broad spectrum of scientific and industrial applications. It can, for example, be used as a nanodevice for drug delivery, delivery of genes for therapy, or the repair of chromosomes. It can be embedded in a membrane or matrix material and serve generally as a portal for translocating polynucleotides from side to the other, as in applications that require moving polynucleotides from one chamber to another.

Optionally, the translocated polynucleotide is linked to a molecular cargo. Molecular cargo that can be translocated from one location to another includes, but is not limited do, one or more polynucleotides. Examples of molecular cargo other than polynucleotides include polypeptides; hormones, drugs, or other small organic molecules; detectable labels; metals; ions; particles; and molecular or multimolecular complexes. The molecular cargo can be covalently or noncovalently (e.g., through base pairing interactions) linked to the polynucleotide.

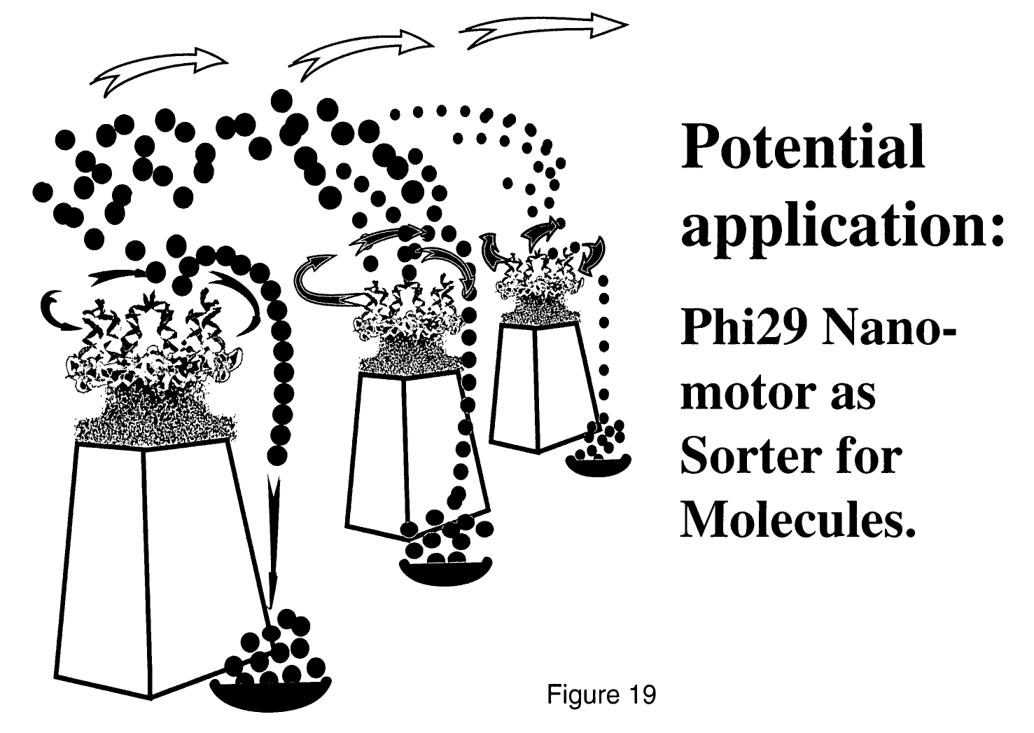

The nanomotor can also be used to perform a sorting function. Advantageously, the 3′ end of the pRNA can be extended by up to about 120 nucleotides without affect pRNA folding and function. The extended sequence can be selected so that it provides as complementary signal to specifically hybridize to a polynucleotide substrate for sorting. For example, a substrate DNA or RNA can be selected based on hybridization to the extended pRNA sequence. The selected polynucleotide is then positioned for translocation by the nanomotor. Since there are six pRNA for each complex, it would be possible to sort up to six different substrates by annealing and denaturation. FIG. 19 illustrates the use of the translocating activity of the nanomotor as a molecular sorter.

Importantly, the nanomotor functions as a molecular pump, which could have a variety of applications in clinical medicine and drug development. Moreover, as a result of its nanoscale size and weight, the nanomotor of the invention is expected to serve as the basis for the development of very strong and light novel materials including nanocomposites, small mechanical devices, and self-assembled biomaterials.

Examples of uses of the nanomotor of the invention include use as a molecular elevator (e.g., J. D. Badjic et al. Science 303: 1845-1848 (2004)), linear shuttle (e.g, P. L. Anelli et al. J. Am. Chem. Soc. 113: 5131 (1991); D. A. Leigh et al. Angew. Chem. Int. Ed. 39: 350 (2000); S. Chia et al. Angew. Chem. Int. Ed. 40: 2447 (2001)), a liquid crystal orientation control device e.g., (R. A. van Delden et al. Proc. Natl. Acad. Sci. U.S.A 99: 4945-4949 (2002)), a muscle, ratchet, pseudorotaxane, or switch (e.g., V. Balzani et al. Acc. Chem. Res. 31: 405-414 (1998); J.-P. Sauvage Acc. Chem. Res. 31: 611-619 (1998); B. L. Feringa et al. Chem. Rev. 100: 1789-1816 (2000); T. R. Kelly et al. Angew. Chem. Int. Ed. 36: 1866-1868 (1997); V. Balzani et al. Angew. Chem. Int. Ed. 39: 3348-3391 (2000); M. C. Jimenez et al. Angew. Chem. Int. Ed. 39: 3284-3287 (2000)). See also C. Bustamante et al. Acc. Chem. Res. 34: 409-522 (2000).

Another application of the nanomotor of the invention is in the development of efficient and sensitive analytical tools that can probe and manipulate single molecules, such as a nanopore-based DNA sequencing devices (D. W. Deamer et al., Trends Biotechnol. 18, 147-151 (2000)). These devices recognize a single base pair, based on the electrical signals generated through the interaction of the bases of the DNA with a pore. A similar concept may be useful for single molecule analysis of other biological molecules. The nanomotor of the invention has the potential to be developed into a DNA-sequencing apparatus, since the DNA-packaging process involves movement of the DNA through a 3.6-nanometer pore surrounded by six RNA that can be modified to accept chemical or electrical signals.

Other Nanoscale Applications

The molecular motor of the invention, as well as components thereof such as pRNA, are well-suited for use as component members of a nanodevice. Nanodevices are structures having dimensions measured in nanometers from about 1-100 nm. These devices are on the same size as biological macromolecules including enzymes and receptors. 50 nm nanodevices can easily enter cells while 20 nm nanodevices can pass out of blood vessels. These devices can be used in biology, chemistry, computer science and electronics, to name just a few technology areas. Nanodevices find medical application as laboratory-based diagnostics as well as in vivo diagnostics and therapeutics, applications which include their use in novel materials, implantable devices, and electrochemical rectifiers, for example.

Nanodevices are expected to play a major role in fighting cancer and other diseases. For example, nanodevices may be used to deliver drugs, such as cancer prevention agents and anti-cancer vaccines, to detect diseased cells, such as cancer cells, through implantable sensors, as contrast agents to determine the location of the cancer within the body, to control the spatial and temporal release of drugs to targeted cells, and to monitor the progress of these drugs.

In addition to the nanoscale components described herein, the nanodevices of the invention may utilize other common biological building blocks for nanoscale ordered structures such as DNA (U.S. Pat. Nos. 5,468,851, 5,948,897, 6,072,044, and WO 01/00876), bacteriophage T even tail fibers (U.S. Pat. Nos. 5,864,013, 5,877,279, and WO 00/77196), self-aligning peptides modeled on human elastin and other fibrous proteins (U.S. Pat. No. 5,969,106), and artificial peptide recognition sequences (U.S. Pat. No. 5,712,366).

Use of pRNA in Nanodevices

DNA lacks structural diversity due to the formation of predominantly double-stranded helices, thus its usefulness in building flexible structures or constructing nanodevices is limited.

In nanodevices of the present invention, another natural type of building block, RNA, is used overcome the limitation of the DNA molecule. Unlike DNA, RNA generally exists in nature as a single-stranded conformation. RNA is in general highly flexible and diverse in structure (A. Mujeeb et al., Nat. Struct. Biol. 5(6): 432 (1998), G. M. Studnicka et al., Nucleic Acids Res. 5: 3365 (1978), D. H. Turner et al., Annu. Rev. Biophys. Chem. 17: 167 (1988), M. Zhong et al., J. Biomolecular Structure & Dynamics 11: 901 (1994), K. Zito et al., Nucleic Acids Res. 21: 5916 (1993), C. C. Correll et al., Cell 91: 705 (1997), A. C. Dock-Bregeon et al., Crystal Structure of a Kinked RNA, in: Molecular Biology of RNA, edited by Liss, New York (1989)).

The astonishing diversity in RNA function is attributed to the flexibility in RNA structure. It has been shown that in most cases it is the structure (i.e., the secondary and tertiary interactions formed by base-pairing within or between single stranded regions), not the primary sequence of RNA that determines its function (C. Chen et al., RNA 5: 805 (1999); T. E. LaGrandeur et al., The EMBO Journal 13: 3945 (1994); D. J. Lane et al., Proc. Natl. Acad. Sci. U.S.A. 82: 6955 (1985)). The primary sequence of RNA gives rise to the 3D structure of RNA that is comprised of helices, bulges, loops, stems, and hairpins (D. H. Turner et al., Annu. Rev. Biophys. Chem. 17, 167 (1988), M. Zhong et al., J. Biomol. Structure & Dynamics 11: 901 (1994), K. Zito et al., Nucleic Acids Res. 21: 5916 (1993), K. Y. Chang et al., J. Mol. Biol. 269(1): 52 (1997), Y. Eguchi et al., J. Mol. Biol. 220: 831 (1991)), however numerous different primary sequences can give rise to the same or essentially same structure if some or all sites of base-pairing interactions are preserved, e.g. via covariation of the bases. Covariation refers to coincident changes in both members of a base pair which preserves base pairing at that position. Indeed, phylogenetic analysis and complementary modification of RNA species have shown that the covariation of bases, if complying with certain rules, can lead to the formation of a defined 3D structure (C. Chen et al., RNA 5: 805 (1999); T. E. LaGrandeur et al., The EMBO Journal 13: 3945 (1994); D. J. Lane et al., Proc. Natl. Acad. Sci. U.S.A. 82: 6955 (1985); S. Bailey et al., J. Biol. Chem. 265: 22365 (1990); E. DeLong et al., Reprint Series 243: 1360 (1989); C. L. Zhang et al., Virology 201: 77 (1994); D. G. Knorre et al., Prog. Nucleic Acid Res. Mol. Biol. 32: 291 (1985)).

pRNA is especially well-suited for use as a component in a nanodevice. As noted herein, the 3′ end of pRNA can be extended up to 120 bases without disrupting motor function. This “extension region” can include additional bases (e.g., ribonucleotides, deoxyribonucleotides, or synthetic analogs thereof), and/or one or more other functional groups, such as a reactive group or a detectable label. The extension region can be used to attach the pRNA, either directly or indirectly, to a substrate so as to immobilize the pRNA, for example to form an array. Alternatively, the extension region can include a capture region to bind molecules of interest. A molecular motor can contain up to six different pRNAs with different (or no) 3′ extension regions.

Importantly, the 3′ extension region can be have a similar function as the “sticky end” of DNA in building branched structures. The availability of a “sticky end” without the disadvantages of the rigid helical structure of DNA, plus the intrinsic property of structure diversity, self-folding, and controllable length, makes pRNA a very attractive component in nanotechnology applications.

Interactions between the pRNA extension region (or other regions of the pRNA) and a substrate or another molecule of interest can be noncovalent or covalent. Examples of nonconvalent interactions include hybridization of the pRNA to a nucleic acid via base pairing interactions, or aptamer-type interactions wherein the pRNA binds to a different type of molecule such as a polypeptide. Covalent linkage of the pRNA to a substrate or other molecule may be facilitated by attaching a reactive group to the extension region, for example by attaching a biotin molecule so as to facilitate interaction with a substrate that has been functionalized with streptavidin. In some embodiments, noncovalent binding interactions between the bound molecule and the pRNA are made covalent by way of, for example, photoactivation.

Circularly permutated pRNA, including pRNA chimeras as described, for example, in U.S. Pat. Publ. 20040126771, published Jul. 1, 2004, can also be used as a component of a nanodevice. A pRNA chimera is formed from a circularly permuted pRNA and a spacer region that includes a reactive group, such as a biologically active moiety. In pRNA chimeras wherein the pRNA region includes or is derived from a naturally occurring pRNA, the spacer region of the pRNA chimera is covalently linked to the pRNA region at what can be considered the “native” 5′ and 3′ ends of a pRNA sequence, thereby joining the native ends of the pRNA region. The pRNA region of the pRNA chimera is optionally truncated when compared to the native bacteriophage pRNA; in those embodiments, and that as a result the “native” 5′ and 3′ ends of the pRNA region simply refer to the nucleotides that terminate or comprise the actual end of the truncated native pRNA. An opening is formed in the pRNA region to linearize the resulting pRNA chimera, effecting a “circular permutation” of the pRNA. It should nonetheless be understood that a circularly permuted pRNA region is not limited to naturally occurring pRNAs that have been circularly permuted but instead is intended to have the broader meaning of RNA having a pRNA-like secondary structure including an opening in the pRNA region that forms the 5′ and 3′ ends of the pRNA chimera, as shown, for example, in FIG. 4 of U.S. Pat. Publ. 20040126771. The reactive group can be incorporated into pRNA for use in diverse applications involving linkage, binding, detection, enzymatic reactions, etc.

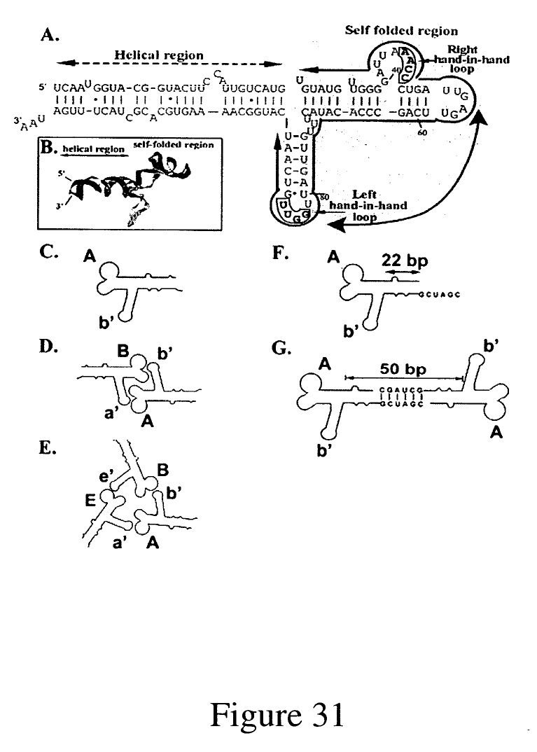

Advantageously, pRNA can manipulated to form monomers, dimers, trimers, hexamers and twins at will, thereby allowing for polyvalent applications (see, e.g., U.S. Pat. Publ. 20040126771, published Jul. 1, 2004, as well as Example III below). A pRNA twin is composed of pRNAs bridged (i.e., linked) via base pairing of a palindromic sequence at the 3′ end of pRNA (see Example IV and FIG. 31G). A homogenous twin is composed of two identical pRNAs, while heterologous twin is composed of two non-identical pRNAs. A “double twin” is a tetrameric structure formed by the complementary loop interactions of two twins. Preferably, the pRNAs used to form a dimer, trimer or hexamers includes the 23/97 segment of pRNA, which segment includes the oligomerization region for the pRNA. Further, as shown below in Example III, pRNA dimers and trimers are typically robust and stable to a wide range of pH from 4-10, a temperature range from −70° C. to 80° C., and to ionic concentrations from 2M NaCl to 2M MgCl₂.

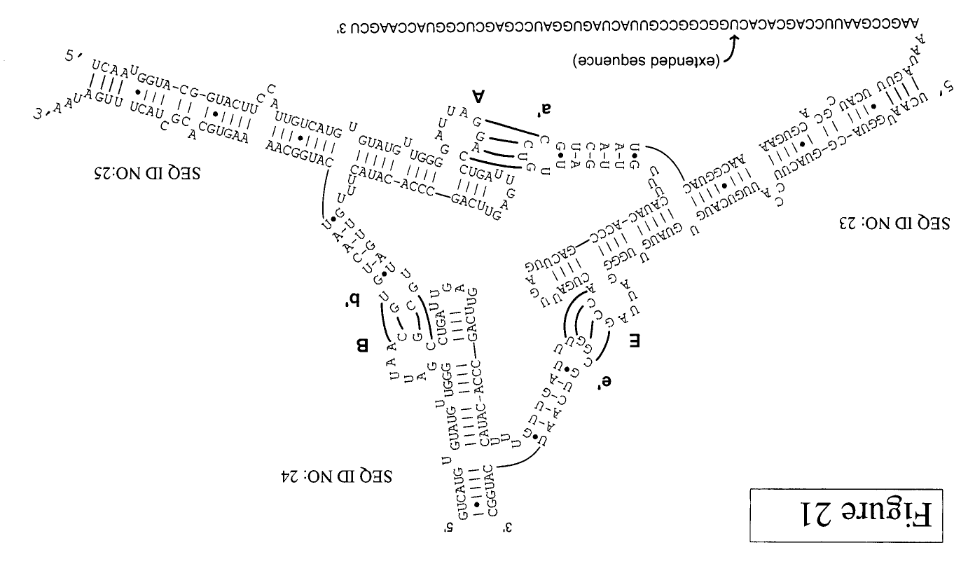

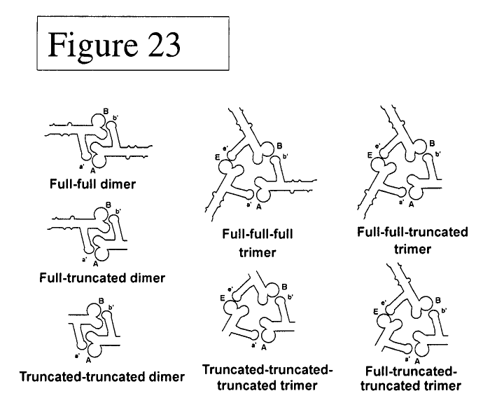

The nomenclature employed to describe the pRNA oligimers is set forth in detail in Example III and is also depicted in FIGS. 21, 22 and 23. Dimerization occurs as a result of complementary interactions of the right and left loops of the pRNA molecule. Uppercase letters are used to describe the right loop of the pRNA and lowercase to represent the left loop. The same letters in upper- and lowercase indicate complementary sequences, whereas different letters mean non-complementary loops. For example, pRNA 5′/3′(A-b′) represents a full-size pRNA with non-complementary right loop A (5′-G⁴⁵GAC) and left loop b′ (3′-U⁸⁵GCG). pRNA complexes can be constructed, for example, from a monomer with intramolecularly self-complementary left and right loops, from monomers with non-complementary left and right loops for intermolecular interaction, and/or from a monomer with intermolecularly self-complementary left and right loops and palindromic 3′ ends. pRNAs useful as reagents and/or nanodevice components could include, for example, monomeric pRNAs having predetermined combinations of right loop, left loop, and 3′ extension regions. The pRNAs can be employed in applications that make use of oligomerization and/or reactivity with the 3′ extension to provide information about the immediate environment of the pRNAs or to achieve a desired result. As a general example, a target molecule may bind one of several pRNAs, each pRNA having a having a different 3′ extension and different right/left loop compositions, such that the identity of the target can be determined by observing the formation of a pRNA oligomer upon contact of another pRNA having complementary loops. The oligomerization event may optionally further trigger the formation of a functional molecular nanomotor.

pRNA Microarrays and Superstructures

Of considerable interest in current nanotechnology is the synthesis of patterned arrays for technological applications. (D. Moll et al., Proc. Natl. Acad. Sci. U.S.A. 99: 14646-14651 (2003), S. L. Burkett et al., Chem. Commun. 3: 321-322 (1996), P. V. Braun et al., Nature 380: 325-328 (1996)). Arrays can be created that serve as chips in the diagnosis of diseases or that function as computerized memory elements. Ordered biological structural arrays can serve as templates for the further construction of superlattices. In particular, nanoarrays can be used to develop diagnostic and therapeutic instruments.

Microarrays can be two-dimensional (2-D) or three-dimensional (3-D) and can be formed from any type of pRNA building block (e.g., monomer, dimer, trimer, tetramer, hexamer, twin, double twin, etc.). pRNA arrays are preferably formed using twin pRNAs. Twins useful in microarrays contain two pRNAs, preferably identical pRNAs, linked by a 3′ palindromic sequence. Preferably, two (e.g., an A-b′ twin and a B-a′ twin) or three (e.g., an A-b′ twin, a B-e′ twin, and an E-a′ twin) twins having intermolecularly complementary loops are preferred for us in forming microarrays.

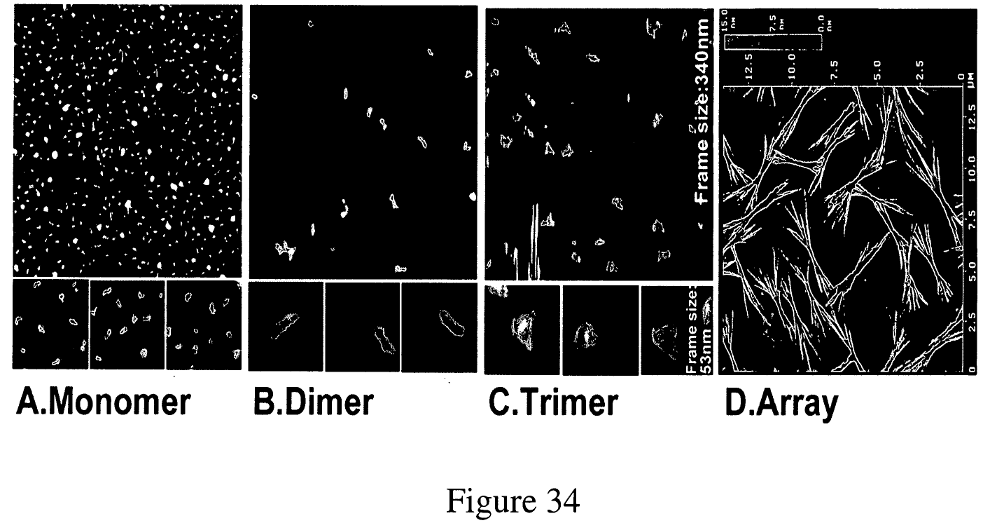

In the wild-type pRNA sequence the helical junction region corresponds to bases 1-28 and 92-117 (see, e.g., FIG. 31A). The pRNA monomers used to form the microarray of the invention preferably have a helical junction region that results in an odd number of half-turns (180°). An odd number of half turns yields a twisting angle of the extending area between the two monomers that allows for continued array growth. As illustrated in Example IV, since each helical turn of RNA is composed of eleven nucleotides, 50 nucleotides (FIG. 31), for example will result, in an odd number of half turns (nine half-turns, or 4.5 turns). When the 50 nucleotides were used as the initial design in array formation, array extension continued successfully (FIG. 34D). The helical region may include one or more bases that are unpaired, such as the bulges shown in the helical region of the wild-type pRNA in FIG. 31A. The left and right loops of the pRNA building blocks aid array growth by continuous extension via loop/loop intermolecular interaction to form a molecular superstructure.

Arrays of pRNA components can be formed in solution, as described in Example IV or attached to a substrate. Preferably, pRNA arrarys are formed in an aqueous environment containing at least 5 mM divalent cation (e.g., Mg⁺⁺, Ca⁺⁺ or Mn⁺⁺) or at least 1 mM monovalent cation (e.g., Na⁺). The arrays are stable at pH from 4 through 12, and temperature ranging from −70° C. to 100° C., and salt concentrations as high as 2M NaCl and 2M MgCl₂.

pRNA molecules can self-assemble into 3-D shapes resembling spirals, triangles, rods and hairpins. From the small shapes that RNA can form (hoops, triangles, etc.) larger more elaborate structures can in turn be constructed, such as rods gathered into spindly, many-pronged bundles. pRNA molecules or higher structures can be used to construct lattices or scaffolding on which complex microscopic machines, such as nano-sized transistors, wires or sensors, can be built and/or mounted. As exemplified in Example IV, the present invention provides a method for controlling the construction of three-dimensional arrays made from RNA building blocks of different shapes and sizes. By designing sets of matching RNA molecules, RNA building blocks can be programmed to bind to each other in precisely defined ways, thereby forming any desired nano-shape. pRNA arrays have many potential applications including specific molecular recognition (e.g., antibodies), molecular sorting, DNA sequencing, and translocation of DNA.

Arrays formed from dimers and trimers are particularly desirable as they can be used as templates to create rod shaped or triangle shaped, respectively, “surface imprints” in a sol-gel matrix or in a polymer film. The ability of these pRNA structures to self-assemble provides the distinct advantage of creating ordered array of imprints in the sol-gel/polymer materials, or gold spray to produce an imprint. These imprinted materials can be used as selective detectors for those particular species.

The structures formed by the dimers and trimers have rod/triangle shaped nanocavities, which can in turn be used for applications such as carrying out electrochemistry and growing metallic, polymer or oxide clusters of varying sizes and dimensions inside the cavities. These structures can be envisaged as potential materials for sensing and biophotonic applications. pRNA hexamers have a cavity or channel of 7.6 nm. which may find application in the transport of biomolecules, for example in a drug delivery system.

### EXAMPLES

The present invention is illustrated by the following examples. It is to be understood that the particular examples, materials, amounts, and procedures are to be interpreted broadly in accordance with the scope and spirit of the invention as set forth herein.

### Example I

### Processive Action of pRNA Drives Bacterial Virus phi29 DNA-Packaging Motor

Materials and Methods

Preparation of pRNA

RNAs were prepared as described in Zhang et al. (1994, Virol. 201, 77-85). Briefly, DNA oligonucleotides were synthesized with the desired sequences and used to produce double-stranded DNA by PCR. The DNA products containing the T7 promoter were cloned into plasmids. RNA was synthesized with T7 RNA polymerase by run-off transcription and purified from a polyacrylamide gel. The sequences of both plasmids and PCR products were confirmed by DNA sequencing.

In Vitro Production of Infectious Virions of phi29 Virion Particles with aptRNA and ATP

The purification of procapsids (Bjornsti et al., 1985, J. Virol. 53(3), 858-861; Vinuela et al., 1976, Philosophical Transactions of the Royal Society of London—Series B: Biological Sciences 276, 29-35), gp16 (Guo et al., 1986, Proc. Nat'l Acad. Sci. USA 83, 3505-3509) and DNA-gp3 (Ortín et al., 1971, Nature New Biol. 234, 275-277), the preparation of the tail protein (gp9) (Garcia et al., 1983, Virology 125, 18-30; Lee et al., 1995, J. Virol. 69, 5018-5023) neck proteins (gp11, gp12) (Carrascosa et al., 1974, FEBS Lett. 44(3), 317-321) the morphogenetic factor (gp13) (Lee et al., 1995, J. Virol. 69, 5018-5023), and the procedure for the assembly of infectious phi29 virion in vitro (Bjornsti et al., 1982, J. Virol. 41, 408-517; (Lee et al., 1995, J. Virol. 69, 5018-5023) were accomplished as previously described.

Briefly, 1 μg of pRNA or its active derivatives (Chen et al., 1999, RNA 5, 805-818; Zhang et al., 1994, Virol. 201, 77-85; Zhang et al., 1997, RNA 3, 315-322), in 1 μl RNase-free H₂O was mixed with 10 μl of purified preformed procapsids (0.4 mg/ml) that devoid of DNA (and dialyzed on a 0.025 μm type VS filter membrane against TBE (2 mM EDTA, 89 mM tris borate/pH 8.0) for 15 minutes at room temperature. The mixture was subsequently transferred for another dialysis against TMS (100 mM NaCl, 10 mM MgCl₂, 50 mM tris/pH 7.8) for an additional 30 minutes.

In the first round, the DNA packaging step, the pRNA-enriched procapsids were then mixed with gp16, DNA-gp3 (a nucleic acid/viral protein covalent chimera that facilitates the translocation of the DNA), and ATP (1.4 mM final concentration except when otherwise indicated) to complete the DNA packaging reaction.

After 30 minutes, in the second round, the assembly step, gp11, gp12, gp9, and gp13, and gp16 were added to the DNA packaging reactions to complete the assembly of infectious virions, which were assayed by standard plaque formation.

Isolation of DNA-Packaging Intermediates and Conversion of the Intermediates into Infectious phi29 Virion

A poorly hydrolyzable ATP analogue, γ-S-ATP, was used in the DNA packaging step (first round) to produce DNA packaging intermediates. DNA-packaging intermediates were generated by the addition of 5% γ-S-ATP (i.e., addition of 1:20 γ-S-ATP:ATP to reach 1.4 mM ATP final concentration) into the phi29 in vitro first round DNA packaging mixture. The intermediates were separated from free DNA and the finished DNA-filled procapsids by 5-20% sucrose gradient sedimentation with SW65 rotor for 30 minutes at 35000 rpm. The gradients were fractionated to separate the components that have different sedimentation rate.

The components in each fraction of the gradient were subsequently converted into infectious phi29 virion by the addition fresh components for phi29 in vitro second round assembly. The complete conversion system (including first and second round components) includes pRNA, gp16, ATP, neck protein gp11/12, and tail protein gp9 and gp13. The infectious virion were titrated by plating on the bacterial host Bacillus subtilis Su⁺⁴⁴.

ATP-Binding Assay for pRNA with ATP-Agarose Affinity Column

A 0.55 cm diameter column was packed with affinity agarose resin (Sigma) immobilized with 1.25-3.25 mM ATP (or other nucleotides) and attached through the C8 (or other position) to cyanogen bromide-activated agarose. Lyophilized resin was soaked in distilled water for more than a half-hour before column packing. After washing with 10 ml of distilled water and then with 10 ml of binding buffer (300 mM NaCl, 20 mM tris/pH 7.6, 5 mM MgCl₂), 1 μg (2.5×10⁻⁵ μmole) of [³H]-labeled RNA in 100 μl binding buffer was applied to the ATP affinity column. The column was then washed with 3 ml of binding buffer, and eluted with the same buffer containing ATP or other nucleotides as indicated. Fractions were collected and subjected to scintillation counting. A 116-base rRNA was used as a negative control.

ATP Gradient Elution to Evaluate the ATP Binding Affinity of pRNA and aptRNA

In ATP gradient elution, a 0.8 cm diameter column was packed with 0.8 ml ATP C-8-agarose immobilized with 1.7 mM ATP. 1 μg (2.5×10⁵ μmole) of [³H]pRNA in 100 μl binding buffer was applied to the column. After washing with 5 ml of binding buffer, RNA was eluted with a 2 ml step-up gradient with increasing concentration of ATP in binding buffer.

Verification of Mutant pRNA Conformation by Competitive Inhibition Analysis

Measurement of binding affinity and virion assembly activity is a reliable and simple method to evaluate conformational changes of mutants with mutations at the location involved in binding. Competitive inhibition assays in combination with binomial distribution were performed to determine the binding affinity. A fixed amount of parental pRNA, pRNA_{wt} or aptRNA was mixed with a varied amount of mutant competitor pRNA in a two-fold serial dilution. Parental pRNA is similar to pRNA_{wt} except that it has two bases at the 5′ and 3′ ends changed to initiate T7 transcription. The “fixed amount” was first determined by titrating a concentration dependant curve of parental pRNA via the plotting of concentration (X-axis) of parental pRNA against the yield of procapsid/pRNA complex (if it is for procapsid binding assay) or virions assembled (if it is used for virion assembly assay). A pRNA concentration required to produce 90% of the maximum yield was taken as the fixed amount of parental pRNA in competitive inhibition analysis.

a. Conformation Verification by Competitive inhibition Assays for Procapsid Binding.

5 ul (2 mg/ml) of purified procapsids in TMS were dialyzed against TBE on a 0.025-um type VS filter membrane at room temperature for 15 minutes. 1 ug of [³H]-parental pRNA (pRNA_{wt} or aptRNA) was mixed with a varied amount of unlabelled competitor RNA in 3 ul of TMS and dried by vacuum. Then the RNAs were resuspended in 5 ul of procapsids that had been dialyzed against TBE for 15 minutes. As a result, the binding volume was limited to 5 ul, and the molar concentration of pRNAs was achieved at a level as high as several uM. After dialysis for another 30 minutes against TMS at room temperature, 95 uL of TMS was added to bring the volume to 100 ul, and the mixtures were then subject to sedimentation via 5-20% sucrose gradient made in TMS to separate procapsid-bound pRNAs from unbound ones. Again, the total cpm of bound [³H]-parental pRNA was plotted against the molar ratio of competitor/total pRNA.

b. Conformation Verification by Competitive Inhibition Assays for phi29 Assembly and the Use of Binomial Distribution to Interpret the Inhibition Curve.

The procedure for using binomial distribution to predict competitive inhibition curves has been described (Trottier et al., 1997, J. Virol. 71, 487-494; Chen et al., 1999, RNA 5, 805-818; Chen et al., 1997, Nucl. Acids Sym. Ser. 36, 190-193). Briefly, in vitro phi29 assembly was performed in the presence of various ratios of parental and mutant pRNAs. The distribution probability of procapsids containing a certain number of mutant and wildtype pRNA was calculated using the binomial equation:

$$\begin{matrix}\begin{matrix}{( p + q )}^{z} = ( \begin{matrix}z \\ 0\end{matrix} ) {p}^{z} + ( \begin{matrix}z \\ 1\end{matrix} ) {p}^{z - 1} q + ( \begin{matrix}z \\ 2\end{matrix} ) {p}^{z - 2} {q}^{2} + … \\ ( \begin{matrix}z \\ z - 1\end{matrix} ) {pq}^{z - 1} + ( \begin{matrix}z \\ z\end{matrix} ) {q}^{z} \\ = {\sum }_{M = 0}^{z} ( \begin{matrix}Z \\ M\end{matrix} ) {p}^{z - M} {q}^{M}\end{matrix} \\ where ( \begin{matrix}Z \\ M\end{matrix} ) is equal to \text{:} ( \frac{Z !}{M ! ( Z - M ) !} ) , \end{matrix}$$

and Z represents the total number of pRNA per procapsid, while p and q represent the % of mutant and parental pRNA, respectively. Since the copy number, Z, of pRNA per procapsid is 6, the expansion of (p+q)⁶ is equal to P⁶+6P⁵q+15P⁴q²+20P³q³+15P²q⁴+6Pq⁵+q⁶. Since p and q are the known number used in assembly, the inhibition curves can be predicted as soon as the activity of parental pRNA has been determined. The probability calculation was extrapolated to predict the yield of pfu/ml produced in each in vitro phi29 assembly reaction. The curves representing the yield of virions from empirical data were plotted against the ratio of mutant pRNA/parental pRNA in the reaction and compared to a predicted curve. If the empirical curve matches the predicted curve, it is an indication that the mutant inactive pRNA had the procapsid binding affinity equal to parental pRNA, that is, the mutant did not change conformation and folding of the pRNA significantly.
ATPase Assay by Thin Layer Chromatography

The purified DNA packaging components gp16 (0.24 μg), DNA-gp3 (0.1 μg), procapsid (3.2 μg) and RNA (1 μg) were mixed, individually or in combination, with 0.3 mM unlabeled ATP and 0.75 μCi (6000 Ci/mmole) [γ-32P]ATP in reaction buffer (Guo et al., 1986, Proc. Nat'l Acad. Sci. USA 83, 3505-3509). When one or more components were omitted, they were replaced with the same volume of TMS. After 30 minutes of incubation at room temperature, 3 μl of the reaction mixture was spotted on to PEI-cellulose plate (J. T. Chem. Co) (Guo et al., 1987, J Mol Biol 197, 229-236) and air-dried. The plate was then soaked in methanol for 5 minutes; air-dried and ran in 1 M formic acid and 0.5 M lithium chloride. Autoradiograms were produced with Cyclone Storage Phosphor Screen. At the same time, a parallel experiment was performed with the same components to test the results of phi29 virion assembly. Only the assembly reactions with the yield higher than 5×10⁷ plaque-forming units per milliliter were selected for ATPase assay.

Results

Isolation of DNA-Packaging Intermediates

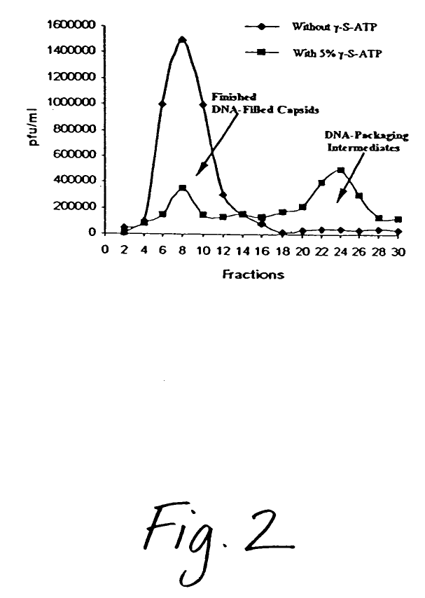

To generate DNA packaging intermediates, the poorly hydrolyzable ATP analog γ-S-ATP was used in the first round packaging reaction. Phi29 DNA packaging was performed in a mixture containing procapsid, gp16, pRNA, genomic DNA-gp3, ATP:γ-S-ATP (1:20), and magnesium. DNA packaging intermediates were separated from free DNA and finished DNA-filled capsids or empty procapsids by sucrose gradient sedimentation. The finished DNA-filled capsids centered at fraction 8 of the gradient (see FIG. 2), while smaller or lighter particles such as free DNA stayed near the top of the gradient. When 5% γ-S-ATP was included in the reaction, significant amounts of DNA-packaging intermediates with smaller sedimentation rates were produced (Fractions 22-26, FIG. 2). DNA packaging was incomplete, and a fragment of the DNA extended from the procapsid. When the ATP included in the DNA-packaging mixture did not include γ-S-ATP, very little DNA packaging intermediates were produced.

After sedimentation, the finished DNA-filled capsids and the DNA packaging intermediates in each fraction of the gradient were converted into mature infectious phi29 virions by the addition of gp16, ATP, neck protein gp11/12, and tail protein gp9. No additional pRNA was added. The resultant infectious virions were titrated by plating on the bacterial host Bacillus subtilis Su⁺⁴⁴.

Both gp16 and pRNA are Required for the Formation of DNA Packaging Intermediates

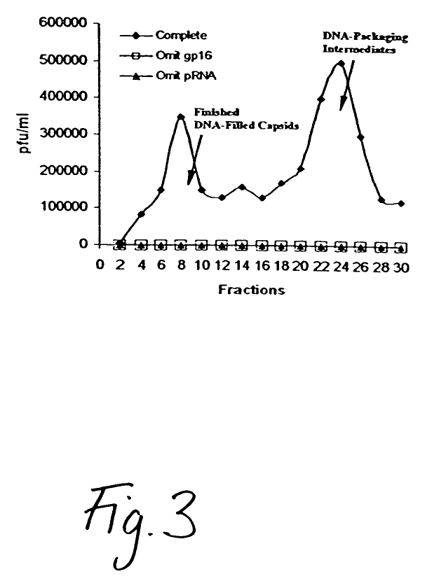

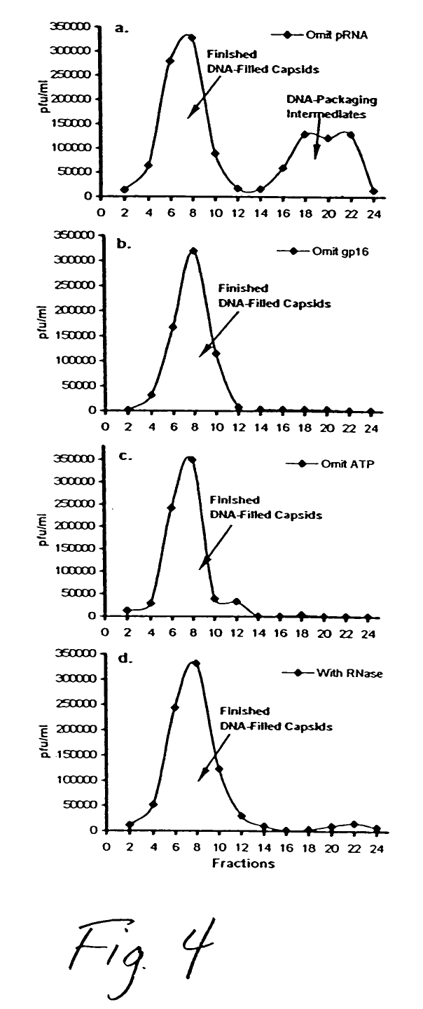

The aforementioned DNA-packaging intermediate isolation method was used to determine which components were necessary for the formation of DNA-packaging intermediates. After sucrose gradient sedimentation of first round packaging reactions including γ-S-ATP, the DNA packaging intermediates were converted in the second round into infectious phi29 virion as described above (FIG. 3). It was found that both gp16 and pRNA were needed for the formation of the intermediates in the first round DNA packaging reaction. If either gp16 or pRNA was omitted from the packaging mixture, no finished DNA-filled capsids or DNA-packaging intermediates were produced in the second round assembly (FIG. 3).

Addition of Fresh gp16 and ATP Molecules to DNA-Packaging Intermediates was Required While Fresh pRNA was not Needed to Convert the DNA-Packaging Intermediates into Finished DNA-Filled Particles

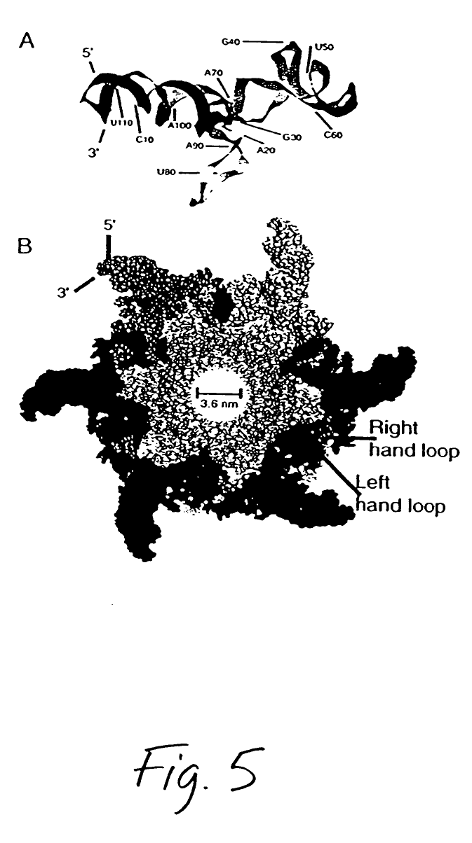

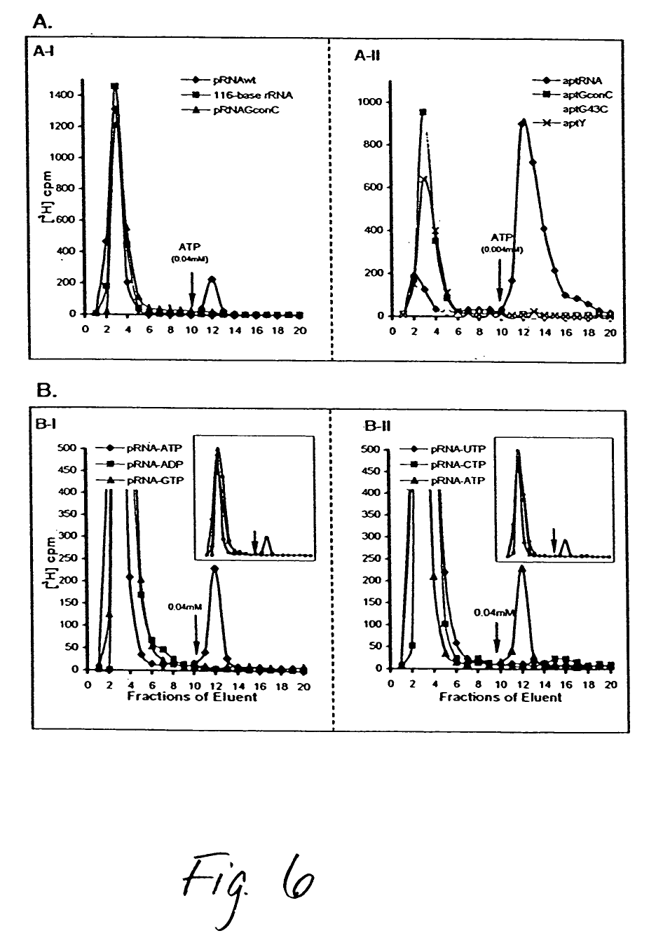

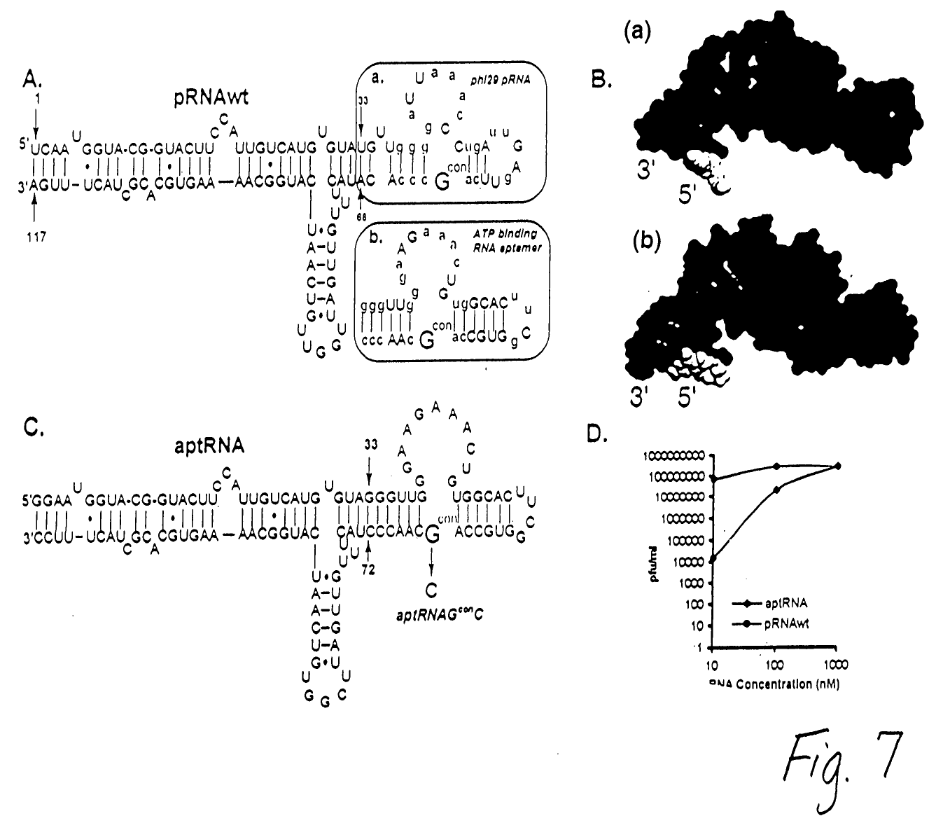

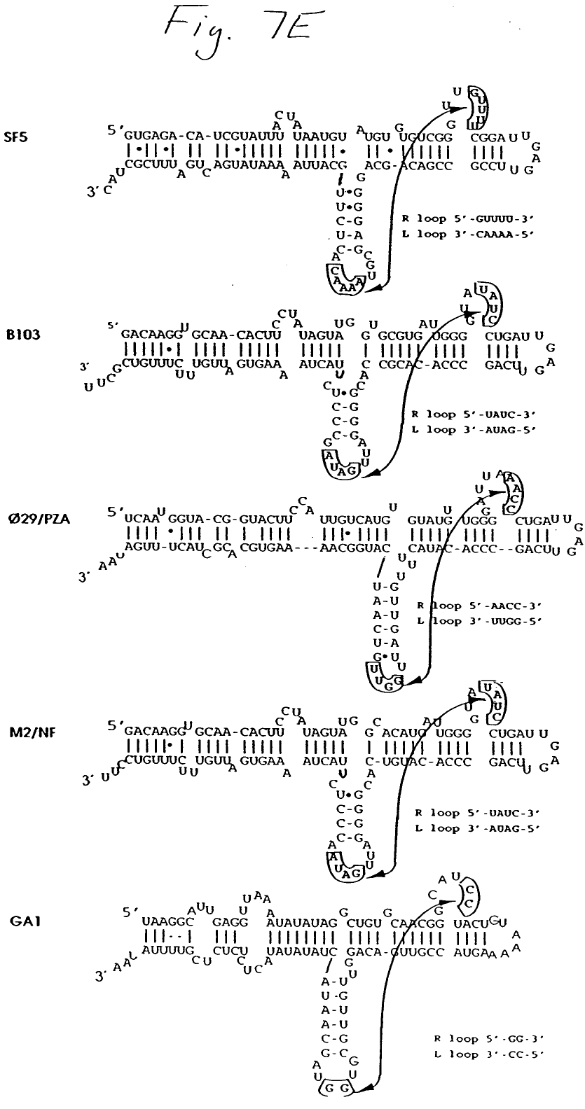

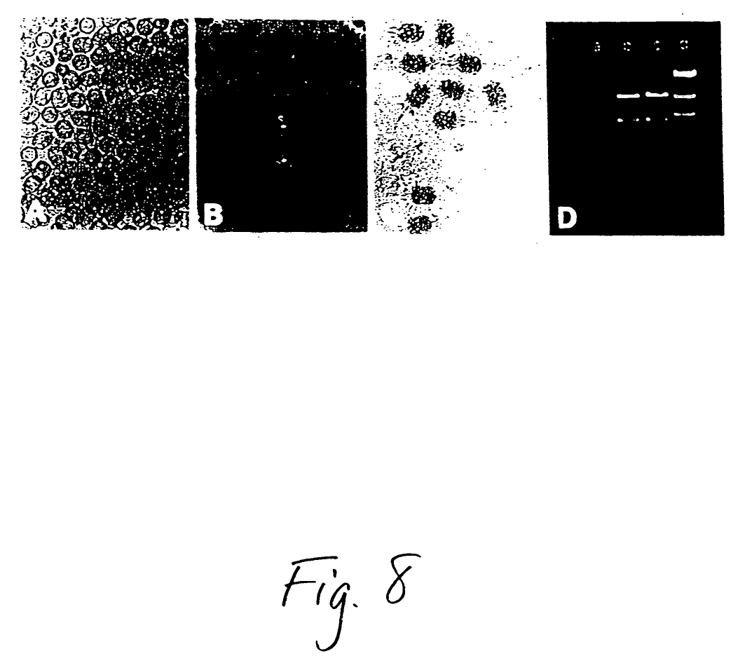

The isolated DNA-packaging intermediates produced from DNA packaging reactions using γ-S-ATP were tested to find out which components are needed to complete the packaging process. ATP, gp16, and pRNA were added individually, or in combination, into each fraction of the gradients in the presence of gp11/gp12 and gp9. It was found that it was not necessary to add pRNA to convert the finished DNA-filled capsid into infectious virion (FIG. 4a), indicating that the binding of six copies of pRNA were sufficient for the continuation of the packaging of the entire DNA genome (FIG. 5). However, it was necessary to add fresh gp16 and ATP to convert DNA-packaging intermediates into infectious phi29 virion (FIGS. 4b and 4c), indicating that the action of gp16 and ATP is not processive. That is, renewed gp16 and ATP were needed during the DNA packaging process and each gp16 and ATP molecule only played a transient role.

The Motor-Bound pRNA was Indispensable During the DNA Translocating Process

It has been reported previously that six pRNA binds to the motor (Guo et al., 1998, Mol. Cell. 2, 149-155; Trottier et al., 1997, J. Virol. 71, 487-494; Zhang et al., 1998, Mol. Cell. 2, 141-147). As already noted, it is not necessary to add fresh pRNA to complete the DNA packaging process. To test whether the procapsid bound pRNA was needed during the DNA translocating process, RNase treatment was conducted to cleave the motor-bound pRNA. It was found that after RNase treatment, the DNA-packaging intermediates could not be converted into infectious virion, while the RNase treatment did not affect the conversion of the finished DNA-filled capsid into infectious virion (FIG. 4d). This is an indication that continued function of pRNA is needed during the DNA translocating process.

Phi29 pRNA was Able to Bind ATP

To investigate whether pRNA could interact with ATP directly, an ATP-agarose affinity column was used to detect the binding of pRNA_{wt,} the shortest pRNA with wildtype pRNA phenotype, to ATP. In FIG. 6, panel A-I, the [³H]pRNA_{wt}, mutant pRNAG^{con}C, and 116-base negative control rRNA were applied onto a 0.8 ml ATP-agarose affinity column and washed with binding buffer. After ten 250-μl fractions, the column was eluted with 0.04 mM ATP in same binding buffer. In FIG. 6, panel A-II, the [³H]aptRNA and other three mutants were tested. [³H]pRNA_{wt} eluted from the column when 0.04 mM ATP was added to the binding buffer, suggesting that pRNA_{wt} binds ATP specifically. When the 116-base rRNA served as the negative control, no detectable RNA was eluted by as high as 5 mM ATP buffer (FIG. 6A-I), thereby indicating that the pRNA/ATP interaction was specific to pRNA. When the conserved base G^{con}, essential for ATP-binding (see below and FIG. 7A), was changed to a C, the resulting mutant pRNAG^{con}C could not bind ATP (FIG. 6A-I) (Table 1).

TABLE 1
ATP-binding and viral assembly activities of pRNA and mutants

| | | | Components added | | | |
|---|---|---|---|---|---|---|
| | | ATP- | | | | Virus |
| | | binding | | | | produced |
| RNAs | Mutation | (%) | ATP | RNase | γ-s-ATP | (pfu/ml) |
| aptRNA | U³³-A⁶⁸ | 80 | − | − | − | 0→ |
| | replaced by | | − | − | + | 0→ |
| | ATP aptamer | | + | + | − | 0→ |
| | | | + | − | + | 0→ |
| | | | + | − | − | 3 × 10⁸ |
| pRNA_{wt} | wild type | 20 | + | − | − | 3 × 10⁸ |
| | pRNA | | | | | |
| aptG^{con}C | G^{con}→C | 0→ | + | − | − | 0→ |
| pRNA_{wt} | G^{con}→C | 0→ | + | − | − | 0→ |
| G^{con}C | | | | | | |
| 116-base | | 0→ | + | − | − | 0→ |
| rRNA | | | | | | |

ATP Binding Affinity of Resins Immobilized with Different Nucleotides or Different Linking Sites

Seven different affinity resins were tested for pRNA_{wt} binding affinity. These resins varied in nucleotide composition and in location for nucleotide/agarose linkage. Our results show that pRNA_{wt} or aptRNA bound only to an agarose resin containing ATP, but not ADP or adenosin-3′,5′-Diphosphate. For ATP resin, pRNA bound only to agarose resins with the attachment site at the C-8 position, but not at N6 or the hydroxyl position. These results suggest that the pRNA_{wt}/ATP interaction requires a specific three-dimensional configuration, and that wild type pRNA_{wt} has a much stronger binding affinity for ATP than for ADP.

Comparison of aptRNA and pRNA_{wt} Binding Affinity to ATP and ADP

It has been reported that in the phi29 DNA packaging system, ATP is hydrolyzed to ADP during packaging (Guo et al., 1987, J Mol Biol 197, 229-236). It would be interesting to know whether pRNA_{wt} can discriminate ATP from ADP. Both ATP and ADP-affinity agarose column immobilized with ATP or ADP, respectively, and attached through the C8 position were used to compare their binding affinity for aptRNA and pRNA_{wt}. As noted earlier, both aptRNA and pRNA_{wt} could attach to ATP-affinity agarose column. However, with the ADP-affinity agarose column, aptRNA or pRNA_{wt} did not bind to the column and passed through the column, appearing only in the first several fractions of the elution. When the ADP column was eluted with 5 mM ADP or ATP, the elution of aptRNA or pRNA_{wt} from the column was almost undetectable, indicating that the binding affinity of aptRNA and pRNA_{wt} to ADP was much lower than that of ATP.

Other approaches for affinity comparison were also made. [³H]aptRNA or [³H]pRNA_{wt} were applied to the ATP-affinity agarose column first, then eluted by ATP or ADP, respectively. Comparison of the elution profiles by ATP and ADP revealed that most of the bound aptRNA and pRNA_{wt} were eluted by 0.004 mM and 0.04 mM ATP, respectively. However, in spite of an expected higher affinity for free ADP then for immobilized ADP (see above), very little aptRNA or pRNA_{wt} was eluted by ADP, even with an ADP concentration as high as 5 mM, supporting the supposition that the binding affinity of aptRNA and pRNA_{wt} to ADP was much lower than that of ATP.

Comparison of RNA Binding Affinity for ATP, CTP, GTP and UTP

To compare the binding affinity for ATP, CTP, GTP and UTP, aptRNA (FIG. 9C) or pRNA_{wt} (FIG. 6B) was first attached to the ATP-agarose gel. After washing with an excess amount of binding buffer, the bound RNA was then eluted by the buffer containing ATP, CTP, GTP and UTP, respectively. It was found that ATP buffer could elute the bound aptRNA or pRNA_{wt} effectively, while GTP, CTP and UTP buffer was much less efficient (FIGS. 6B and 9C).

The Central Region of phi29 pRNA is Very Similar to ATP-Binding RNA Aptamer in Both Sequence and Predicted Secondary Structure.

A chemically selected aptamer RNA has been found to be able to bind ATP (Sassanfar et al., 1993, Nature 364, 550-553) (FIG. 7A-b). The structural basis for this ATP-binding RNA aptamer has also been elucidated by multidimensional NMR spectroscopy (Cech et al., 1996, RNA 2, 625-627; Dieckmann et al., 1996, RNA 2, 628-640; Jiang et al., 1996, Nature 382, 183-186). (FIG. 7B-d). All ATP-binding aptamers contain a consensus sequence embedded in a common secondary structure (Cech et al., 1996, RNA 2, 625-627; Dieckmann et al., 1996, RNA 2, 628-640; Sassanfar et al., 1993, Nature 364, 550-553; Jiang et al., 1996, Nature 382, 183-186). The bases essential for ATP-binding have been identified (Sassanfar et al., 1993, Nature 364, 550-553; Jiang et al., 1996, Nature 382, 183-186). The structure of the phi29 pRNA has been investigated extensively (for review, see Guo, 2002, Prog. Nucl. Acid Res. & Mol. Biol. 72, 415-473). It would be intriguing to investigate whether the chemically selected ATP-binding RNA moiety is present in a living system. We compared the structure of ATP-binding aptamers with phi29 pRNA and found that the ATP-binding RNA aptamer is very similar to the middle part of phi29 pRNA (FIG. 7B-c) in both sequence and structure (FIGS. 7A&B).

Infectious Virus was Produced in the Presence of the Chimeric aptRNA Harboring the ATP-Binding Moiety

To further confirm that an ATP-binding moiety is present in a pRNA molecule, the pRNA moiety with a potential for ATP-binding was replaced with an ATP-binding RNA aptamer, ATP-40-1 (Sassanfar et al., 1993, Nature 364, 550-553). A chimeric aptRNA was constructed by replacing bases 33-68 (36 bases) with the sequence of ATP-40-1 (40 bases) (Sassanfar et al., 1993, Nature 364, 550-553; Jiang et al., 1996, Nature 382, 183-186; Cech et al., 1996, RNA 2, 625-627). (FIG. 7-C). When the chimeric aptRNA was added to the phi29 in vitro assembling mixture (Lee et al., 1994, Virol, 202, 1039-1042; Lee et al., 1995, J. Virol. 69, 5018-5023) about 10⁸ infectious virus particles per milliliter were produced in the test tube (Table 1, FIG. 8). Omission of ATP or aptRNA, or the addition of RNase to the reaction mixture, failed to generate a single virus (Table 1).

ATP is Required for the Production of Infectious Virus

To establish that the activity of aptRNA is related to ATP, virus assembly using aptRNA was performed with and without the presence of ATP. When ATP was omitted from the reaction, not a single plaque was detected. Virus assembly was also inhibited by the poorly hydrolysable ATP analogue γ-S-ATP, suggesting that the aptRNA-involved viral assembly process is ATP related (Table 1).

AptRNA Bound ATP

An ATP-affinity agarose column was used to detect whether the aptRNA could bind ATP. [³H]RNA was applied to an ATP affinity column. [³H]-aptRNA was found to bind to the ATP matrix and did not run through the column (FIG. 6A-II). Additionally, aptRNA was eluted from the column with 0.004 mM ATP, suggesting that the binding of aptRNA to the column is due to specific ATP and aptRNA interaction. The 116-base rRNA negative control did not bind to the column (FIG. 6A-I).

ATP-Binding Affinity for pRNA and aptRNA

The ATP binding affinity of both RNAs were evaluated by ATP gradient elution. Free ATP (ATP_{free}) will compete with the column-bond ATP (ATP_{bound}) for binding to aptRNA or pRNA_{wt}. From the ATP gradient elution (FIG. 9), it was found that most of the bound aptRNA and pRNA_{wt} was eluted by 0.004 mM and 0.04 mM ATP_{free}, respectively.

Changing of a Single Base Essential for ATP Binding Abolished Both the ATP-Binding and Viral Assembly Activities

Nucleotide G^{con} (FIG. 7) has been shown to be highly conserved in ATP-binding RNA aptamers, and is the most critical nucleotide for ATP binding (Dieckmann et al., 1996, RNA 2, 628-640; Sassanfar et al., 1993, Nature 364, 550-553). One G corresponding to G^{con} of the aptRNA is also conserved in all the pRNAs of five different bacteriophages (Bailey et al., 1990, J. Biol. Chem. 265, 22365-22370; Chen et al., 1999, RNA 5, 805-818).

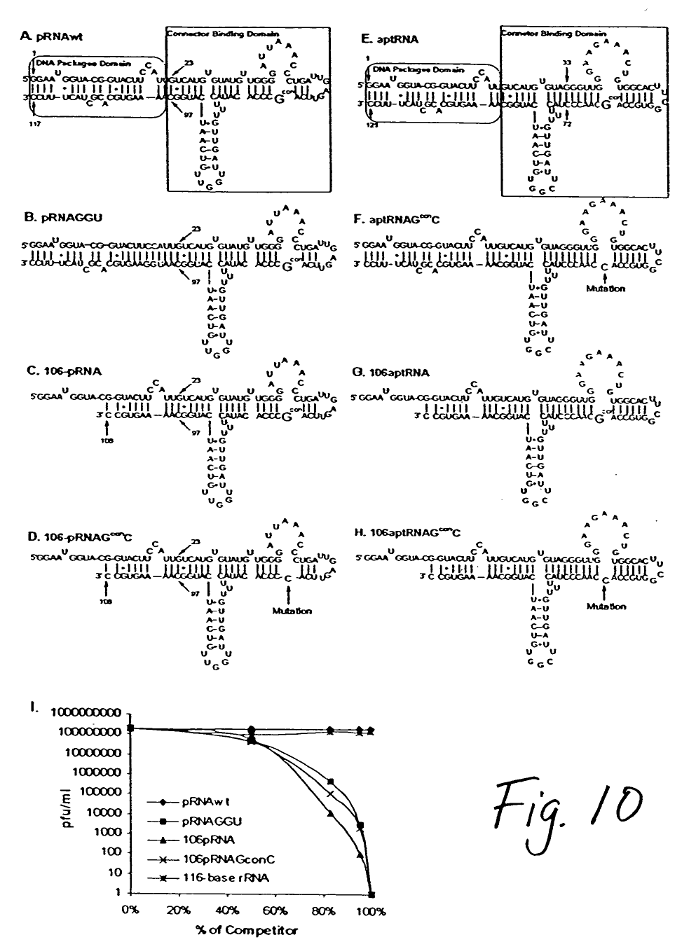

Mutation of G^{con} to C resulted in a mutant aptG^{con}C (FIG. 10F) that was not able to bind ATP (FIG. 6A-I). This mutant was also completely inactive in virion assembly (Table 1), suggesting that the functions of ATP-binding and virion assembly are correlated. When the G^{con} mutation was introduced into the conserved G^{con} of wild type pRNA, the ATP-binding activity of the mutant pRNAG^{con}C disappeared (FIG. 6). This mutant was found to be incompetent in phi29 assembly (Table 1).

Verification of Conformation and Folding After the Change of One Single Base Essential for ATP Binding

As noted above, a single base mutation completely obliterated the activity of pRNA_{wt} and aptRNA in both ATP-binding and virion assembly. To confirm that the loss of activity in such a single base mutation is due to the change of pRNA chemistry rather than to the change in conformation or folding, competitive inhibition assays were performed (see Materials and Methods) to test whether the conformation of the mutant RNA is identical to its parental pRNA.

Two pRNAs, 106-pRNA and 106-pRNAG^{con}C (FIG. 10), were used for structural comparison. In these two pRNAs, eleven nucleotides from G¹⁰⁷ to C¹¹⁷ in the DNA translocating domain were deleted. It has previously been shown that the deletion of these eleven nucleotides did not affect the connector binding affinity of the resulting mutants (Trottier et al., 1997, J. Virol. 71, 487-494; Trottier et al., 1996, J. Virol. 70, 55-61; Garver et al., 1997, RNA 3, 1068-1079; Chen et al., 1999, RNA 5, 805-818). If the change of G^{con} to C would change the conformation or folding of the mutant pRNA, the resulting mutants 106-pRNAG^{con}C will not be able to compete with its parental pRNA_{wt} for binding to procapsid or other substrates, and thus will not be able to inhibit the parental pRNA_{wt} for procapsid binding, DNA packaging and phi29 assembly.

Competitive inhibition analysis revealed that 106-pRNAG^{con}C mutants were able to compete with pRNA_{wt} for procapsid binding and inhibit the assembly of phi29 virions (FIG. 9). Comparison of inhibition curves (FIG. 10-I) revealed that the inhibition efficiency of 106-pRNAG^{con}C is very similar to the control 106-pRNA as well as pRNAGGU, that has been shown to maintain wild type conformation in the procapsid binding domain (Chen et al., 1999, RNA 5, 805-818; Zhang et al., 1997, RNA 3, 315-322). Therefore, it can be concluded that the changing of G^{con} to C did not cause a conformational change in the resulting mutant pRNAs in relation to procapsid binding. Competitive inhibition analysis also revealed that the inhibition profile of mutant 106aptRNAG^{con}C is very similar to that of 106aptRNA (FIGS. 10H and G), supporting the conclusion that the changing of G^{con} to C did not cause a significant conformational change.

Conformational Changes of pRNA Induced by ATP During Packaging

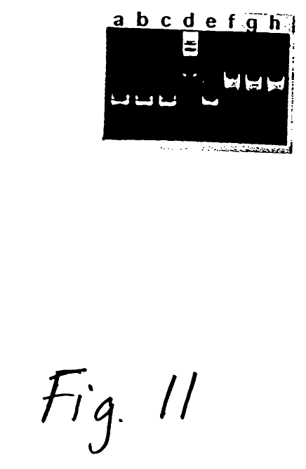

The conformation change of pRNA_{wt} was investigated in the presence and absence of ATP. ATP caused a change in the pRNA_{wt} migration rate in native gels (FIG. 11). Purified pRNA_{wt} was loaded onto an 8% native polyacrylamide gel (Chen et al., 2000, J. Biol. Chem. 275(23), 17510-17516) with increasing concentrations of ATP. A pRNA band shift was observed in the presence of ATP (lane f-h), but not observed in the absence of ATP (lane e), while the 5S rRNA control did not show any migration change either in the presence (lanes b-c) or absence (lane a) of ATP. The band with the slower migration rate was purified and shown to be fully active in DNA packaging. At the same time, control E. coli 5S rRNA did not show any migration rate change due to the presence or absence of ATP (FIG. 11).

We have previously reported that pRNA formed oligomers with slower migration rate in gel when magnesium is present (Guo et al., 1998, Mol. Cell. 2, 149-155). Chen et al., 2000, J. Biol. Chem. 275(23), 17510-17516) The formation of a band with a slower migration rate in FIG. 11 suggests that, in the presence of ATP, the conformation or oligomerization of pRNA is larger rather than smaller. This phenomenon argues against the possibility that the change of pRNA conformation is due to the depletion of ion by ATP. If that were true, the RNA should become smaller and run faster in the presence of ATP. The appearance of a broad band representing pRNA with a slower migration rate also suggests that more than one conformation of pRNA may be present, or that the pRNA/ATP complex is relatively unstable.

ATP was Hydrolyzed to ADP and Inorganic Phosphate in a Reaction Mixture pRNA

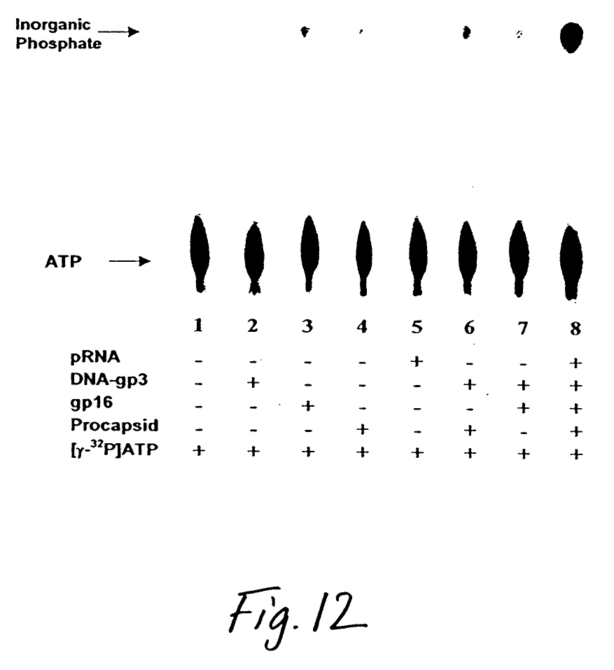

Hydrolysis of [³²P]-ATP was assayed by thin layer chromatography on a PEI-cellulose plate. Components involved in DNA packaging were mixed, alone or in combination, with [³²P]-ATP. After an incubation period of 30 minutes, the reaction mixture was applied to the PEI-plate. Results from thin layer chromatography revealed that the individual component alone or in combination without the presence of pRNA (FIG. 12), exhibited low undetectable ATPase activity. However, ATP was hydrolyzed to inorganic phosphate in the reaction including pRNA.

Discussion

To secure the continuous motion of the nanomotor, at least one component should act processively to keep the motor drive continually. In bacterial virus phi29, the DNA-packaging motor is composed of the connector, gp16 and ATP. The connector is excluded from the candidate list of processive factor, since the crystal structure of connector reveals no potential ATP-binding pocket. Gp16 and pRNA are the only candidates for this processive factor.

Our results showed that both gp16 and pRNA are not needed to convert the finished DNA-filled capsids into infectious viruses (FIGS. 2, 3 and 4). This is comprehensible since the DNA-packaging in these particles has been completed. However, to convert the partially filled DNA-packaging intermediates into completed DNA-filled particles, fresh gp16 and ATP but not pRNA are needed (FIG. 4). This is an indication that multiple copies of fresh gp16 and ATP have to jump in to join the DNA translocating process. However, the six copies of pRNA that have already bound to the motor are sufficient to complete the DNA packaging work. In addition, pRNA were working during the DNA packaging process, since when the intermediates were treated with RNase, DNA-packaging in intermediates could not be completed and no infectious virus was produced from the intermediate (FIG. 4d). In combination with the fact that pRNA could bind ATP, it is predicted that pRNA is the processive factor in phi29 DNA packaging motor.

It has also been shown that six copies of pRNA bind to the connector (Trottier et al., 1997, J. Virol. 71, 487-494; Zhang et al., 1998, Mol. Cell. 2, 141-147, Hendrix, 1998, Cell 94, 147-150; Guo et al., 1998, Mol. Cell. 2, 149-155) that is embedded in an icosahedral protein shell that has a five-fold rotational symmetry (Simpson et al., 2000, Nature 408, 745-750; Jimenez et al., 1986, Science 232, 1113-1115). If the nanomotor indeed rotates, then the setting of the hexameric pRNA within a 5-fold symmetrical environment could constitute a mechanical apparatus with two symmetrically mismatched rings that will produce a continuous rotating force in order to drive the motor (Chen et. al., 1997, J. Virol. 71, 3864-3871; Hendrix, 1978, Proc. Natl. Acad. Sci. USA 75, 4779-4783). Conformational change of molecules induced by ATP is a common phenomenon in biosystems, such as myosin, kinesin, helicase and RNA polymerase that involve motion. Our finding that ATP induced a conformational change of pRNA might boost a speculation that pRNA is part of the driving force, displaying contraction and relaxation as proposed previously (Chen et al., 1997, J. Virol. 71, 3864-3871).

Mutation studies of pRNA_{wt} and aptRNA have revealed that, within each pRNA_{wt} or aptRNA group, ATP-binding affinity is correlated to phi29 virion assembly (Table 1). However, outside the group, this correlation could not apply. For example, the ATP-binding affinity of aptRNA is stronger than pRNA_{wt}, but the viral assembly activity of aptRNA is not higher than pRNA_{wt} (FIG. 8D). Three possibilities might explain this discrepancy. First, the binding of pRNA to the connector is the rate determining step in phi29 DNA packaging and assembly. A 29-base change in the connector-binding domain of aptRNA might somehow alter its structure and thus hamper the connector binding affinity. As shown in FIG. 8D, the concentration requirement to reach a 50% plateau of the assembly curve for aptRNA is higher than for pRNA_{wt}. This is an indication that the binding affinity (Kₐ) of aptRNA/connector complex is lower than that of pRNA_{wt}/connector complex. Second, although the chemically selected ATP-binding aptamer is an excellent molecule for ATP binding, it might not be, after all, the best candidate in nature for ATP hydrolysis if such hydrolysis does occur. Third, too high a binding affinity to the substrate does not signify a good enzyme, since this enzyme will not be dissociated from its substrate easily. Such dissociation might be critical for the turnover in pRNA/ATP interaction in phi29 assembly.

Here we found that the putative ATP-binding site in pRNA resides within a region interacting with the connector. The significance for such ATP/pRNA binding remains to be investigated. One possible implication is that ATP binding to pRNA provides a special structure in the assembly of the packaging machinery. Another possible implication is that alternative binding and release of ATP from pRNA could induce a conformational change of pRNA that in turn rotates the connector.

### Example II

### Construction of a Controllable 30-nm Nanomotor Driven by a Synthetic ATP-Binding RNA

Experimental Procedures

Synthesis of aptRNA

AptRNA (FIG. 7C) was synthesized both chemically and enzymatically. With the chemical method, an additional ligation step was used to synthesize the 121-base aptRNA from smaller synthetic RNA oligonucleotides. With the enzymatic method, RNA was synthesized with T7 RNA polymerase by run-off transcription and purified from a polyacrylamide gel. The sequences of both plasmids and PCR products were confirmed by DNA sequencing. No difference in DNA-translocation and viral assembly activity was found with RNA from both methods.

In Vitro Construction of the Nanomotor and Testing of Motor Function by its Ability to Produce Infectious phi29 Virion.

Procapsids and gp16, as well as the phi29 structural proteins gp9, gp11 and gp12 were purified from products of genes that were cloned into plasmid. pRNA enriched procapsids were synthesized as in Example I. The pRNA-enriched procapsids were then mixed with purified gp16, DNA, and ATP to complete the DNA packaging reaction (the first round, DNA packaging). After 30 minutes, gp11, gp12, and gp9, gp13, and fresh gp16 were added to the DNA packaging reactions in the second round (phage assembly) to complete the assembly of infectious virions, which were assayed by standard plaque formation.

Testing for Turning Off and on of the Motor Function

The motor was turned off by the addition of ATP analogue, and the DNA-packaging intermediates with partially packaged DNA were isolated due to the halting of the motor. The turned off motor was turned on again by the addition of ATP and assayed for the production of infectious virion. DNA-packaging intermediates were isolated and converted into infectious phage as in Example I.

ATP-Binding Assay for pRNA with ATP-Agarose Affinity Column

ATP binding of aptRNA and related molecules was accomplished as in Example I.

Gel Shift Assay

Purified aptRNA was loaded onto an 8% native polyacrylamide gel with an increasing amount of ATP. A 5S rRNA was used as a control.

Determination of Apparent Dissociation Constants (K_{D},app) for aptRNA/ATP Complex.

The K_{D,app} for RNA/ATP interaction was determined by the methods of isocratic elution and ATP gradient elution. The isocratic elution method was used to measure the K_{D,app} for ATP that immobilized on agarose (ATP_{bound}), while the method of ATP gradient elution was to measure the K_{D,app} for free ATP (ATP_{free}).

Isocratic elution. [³H]aptRNA was applied to a column (0.55 cm in diameter) packed with ATP-C-8 affinity agarose (2.7 ml) and eluted with binding buffer. Fractions (2 ml) were collected and subjected to scintillation counting. K_{D,app} was determined with the equation: K_{D,app}=[L]×(V₁−V₀)/(Vₑ−V₀) where [L] is the concentration of ATP immobilized on agarose (1.7 mM), V₁ is the volume of the column (2.7 ml), V₀ is the void volume of the column (2.09 ml), and Vₑ is the volume needed to elute the RNA (32 ml). The K_{D,app} for aptRNA interacting with the ATP_{bound} was determined to be 0.035 mM.

ATP gradient elution. [³H]aptRNA was applied to a column (0.55 cm in diameter) packed with ATP-C-8 affinity agarose (0.8 ml) and eluted with a 2 ml step-up gradient with a specified concentration of ATP in binding buffer. Fractions were collected and subjected to scintillation counting. The K_{D,app} for the complex of aptRNA/ATP_{free} is around 0.004 mM.

Results

Infectious Viruses were Produced in the Test Tube Using the Artificial aptRNA

The gene coding for the three bacterial virus phi29 protein components gp7, gp8 and gp10 that are needed for building a functional virus were cloned into plasmid and transformed into E. coli cells The particles assembled in E. coli were similar to phi29 procapsids. The purified particles from E. coli were then incubated with the synthetic aptRNA, which automatically bound to the particles. In the presence of ATP, this RNA could power a motor to rotate and move the 19 Kbp-phi29 genomic DNA into the protein shell to produce infectious viral particles in vitro with a titer of 10⁸ infectious virus particles per milliliter (Table 2, FIG. 8). Omission of ATP or aptRNA or the addition of RNase to the reaction mixture failed to generate a single virus (Table 2).

TABLE 2
Production of Infectious virus with aptRNA and ATP

| | |
|---|---|
| | Virus |
| Components | produced |

| AptRNA | ATP | RNase | γ-S-ATP | (pfu/ml) |
|---|---|---|---|---|
| + | + | − | − | 2 × 10⁸ |
| − | + | − | − | →0 |
| + | − | − | − | →0 |
| + | + | + | − | →0 |
| + | + | − | + | →0 |

AptRNA Bound ATP

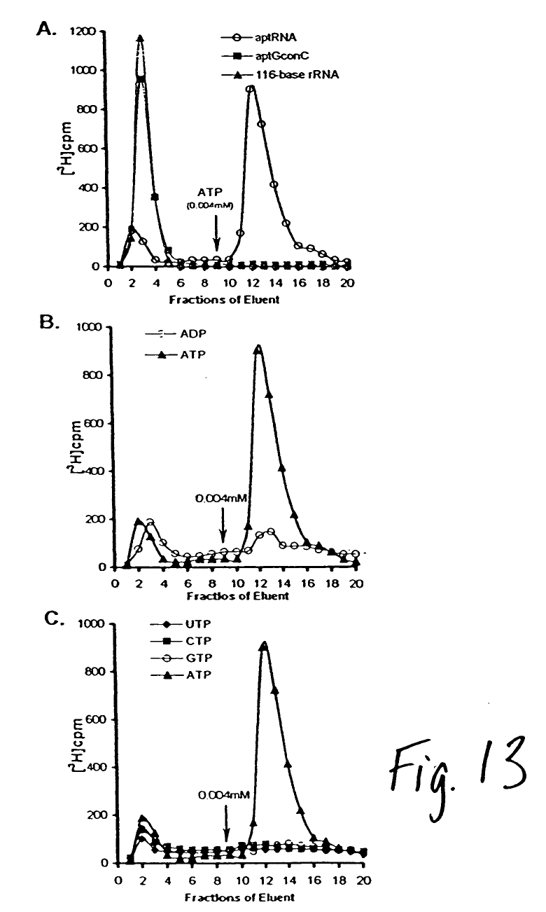

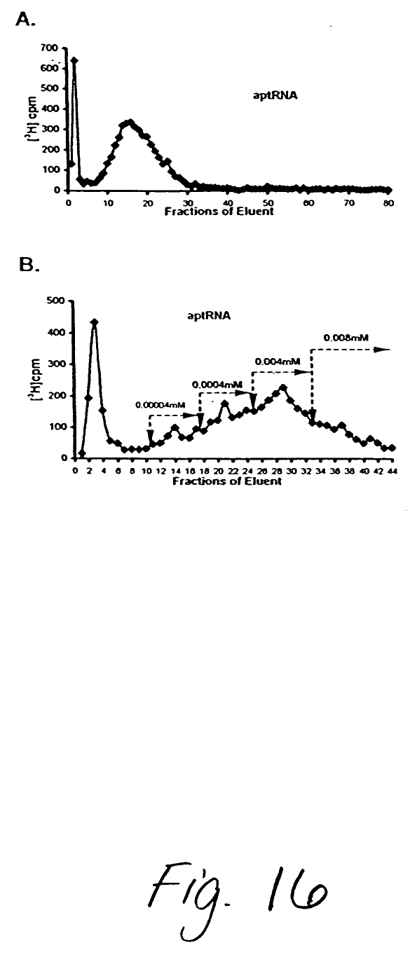

An ATP-affinity agarose column was used to detect whether the aptRNA could bind ATP. [³H]RNA was applied to an ATP affinity column. Most [³H]aptRNA was found to bind to the ATP matrix and did not run through the column (FIG. 13). Additionally, aptRNA was eluted from the column with 0.004 mM ATP, suggesting that the binding of aptRNA to the column is due to specific ATP and aptRNA interaction. AptRNA was not eluted by ADP, UTP, CTP or GTP (FIG. 13 B,C). The 116-base rRNA negative control did not bind to the column (FIG. 13A). The K_{D,app} for the RNA/ATP interaction was determined to be 0.035 mM for resin-bound ATP and 0.004 mM for free ATP (FIG. 16). The finding of a difference in the K_{D},app determined via these two methods is not surprising because the C-8 linkage of ATP to agarose might hamper the RNA/ATP interaction that involves a three-dimensional contact. Furthermore, it is possible that only a certain fraction of ATP_{bound} in the gel is accessible to aptRNA.

Comparison of aptRNA Binding Affinity to ATP and ADP

In bio-systems, energy is derived from the hydrolysis of ATP to ADP. It would be interesting to know whether aptRNA can discriminate ATP from ADP. Both ATP and ADP-affinity agarose columns were immobilized with ATP or ADP, respectively, and attachments through the C8 position were used to compare their binding affinity for aptRNA. As noted earlier, aptRNA could attach to an ATP-affinity agarose column. However, when aptRNA was applied to the ADP-column, most of the aptRNA did not bind to the column but passed through, appearing only in the first several fractions of the elution. When the ADP column was eluted with 4 mM ADP or ATP, the elution of aptRNA from the ADP column was very low. The concentration used here was 1000-fold higher than that used for the ATP column, indicating that the binding affinity of aptRNA to ADP was much lower than that of ATP.

Other approaches for affinity comparison were also made. [³H]aptRNA was applied to the ATP-affinity agarose column first, then eluted by ATP and ADP, respectively. Comparison of the elution profiles by ATP and ADP revealed that most of the bound aptRNA was eluted by 0.004 mM ATP. However, in spite of an expected higher affinity for free ADP than for immobilized ADP, very little aptRNA was eluted by ADP (FIG. 13B), even with an ADP concentration as high as 5 mM, supporting the conclusion that the binding affinity of aptRNA to ADP was much lower than that of ATP.

Comparison of AptRNA Binding Affinity for ATP, CTP, GTP and UTP

AptRNA was first attached to the ATP-agarose gel. After washing with an excess amount of binding buffer, the bound RNA was then eluted by buffers containing ATP, CTP, GTP and UTP, respectively. It was found that the ATP buffer could elute the bound aptRNA effectively, while the GTP, CTP and UTP buffers were much less efficient (FIG. 13C).

Changing of a Single Base Essential for ATP Binding Abolished Both the ATP-Binding and Viral Assembly Activities

The structural basis for ATP-binding RNA aptamers has also been clarified by multidimensional NMR spectroscopy. All ATP-binding aptamers contain a consensus sequence embedded in a common secondary structure and the bases essential for ATP-binding have been identified. Nucleotide G^{con} (Example I, FIG. 7C) has been shown to be highly conserved in ATP-binding RNA aptamers and is the most critical nucleotide for ATP binding.

Mutation of G^{con} to C resulted in a mutant aptG^{con}C (Example I, FIG. 7C) that was not able to bind ATP (FIG. 13A). This mutant was also completely inactive in virus assembly (Table 3), suggesting that the functions of ATP-binding and virus assembly are correlated. By structural analysis, in addition to competition and inhibition with binomial distribution analysis, it was confirmed that the incompetence of such mutant aptRNA in motor driving is due to a change in chemistry rather than structure.

TABLE 3
Activities of aptRNA and Mutant

| | | | Virus produced |
|---|---|---|---|
| RNAs | Mutation | ATP-binding | (pfu/ml) |
| aptRNA | none | + | 10⁸ |
| aptG^{con}C | G^{con}→C | − | →0 |
| 116-base | N/A | − | →0 |
| rRNA | | | |

ATP is Required for the Production of Infectious Virus

To establish that the activity of aptRNA is related to ATP, virus assembly using aptRNA was performed with and without the presence of ATP. When ATP was omitted from the reaction, not a single plaque was detected. Virus assembly was also inhibited by the poorly hydrolysable ATP analogue γ-S-ATP, suggesting that the aptRNA-involved viral assembly process is ATP related (Table 2).

Conformational Changes of the ATP-Binding RNA Induced by ATP

In the mechanism of the movement of muscle, alternative binding and release of ATP induces a conformational change of the muscle to produce a transition. Does aptRNA move by conformational change induced by ATP? The change in conformation of the ATP-binding RNA was investigated both in the presence and absence of ATP using a gel shift assay. Purified ATP-binding RNA was loaded onto a native gel with increasing concentrations of ATP. ATP caused a change in the RNA migration rate in native gels (FIG. 11). The ATP-binding RNA was observed to migrate slower when ATP was present. A band shift of ATP-binding RNA was observed in the presence of ATP (lane f-h), but not observed in the absence of ATP (lane e), while the 5S rRNA control did not show any migration change either in the presence (lanes b-c) or absence (lane a) of ATP. The band with the slower migration rate was purified and shown to be fully active in DNA packaging. At the same time, a control E. coli 5S rRNA did not show any migration rate change in the presence or absence of ATP (FIG. 11).

It has previously been reported that pRNA formed oligomers with a slower migration rate in gel when magnesium was present. The formation of a band with a slower migration rate in FIG. 11 suggests that, in the presence of ATP, the conformation or oligomerization of pRNA is larger rather than smaller. This phenomenon argues against the possibility that the change of ATP-binding conformation is due to the depletion of an ion by ATP, but in favor of a speculation that ATP induces RNA conformational changes. If that were true, the RNA should become smaller and run faster in the presence of ATP. The appearance of a broad band representing ATP-binding RNA with a slower migration rate also suggests that more than one conformation of ATP-binding RNA may be present, or that the RNA/ATP complex is relatively unstable.

ATP was Hydrolyzed to ADP and Inorganic Phosphate in a Reaction Mixture with aptRNA

To assay for ATPase activity, components involved in DNA packaging were mixed alone, or in combination, with [³²P]ATP. Results from thin layer chromatography revealed that the individual components alone, or in combination without the presence of aptRNA (FIG. 12), exhibited low undetectable ATPase activity. However, ATP was hydrolyzed to inorganic phosphate in the reaction including aptRNA.

Motor Could be Turned Off by EDTA, γ-S-ATP and RNase

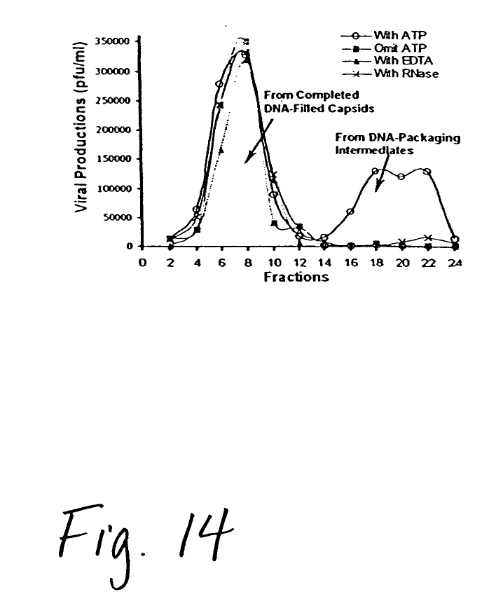

One of the important issues in constructing a viable molecular motor or shuttle involves how to switch it on and off. It was shown that this DNA-packaging motor could be turned off with the addition of EDTA, RNase (FIG. 14), or poorly hydrolyzable ATP analogues, such as γ-S-ATP (FIG. 2, Example I).

The Turned-Off Motor Can be Started Again by ATP or Magnesium, but is Irreversible if Shut Off by RNase

A usable motor must be able to run again after being shut off. To test whether the stationary motor turned off by EDTA, RNase or γ-S-ATP could be switched on again, the intermediates containing blocked motors were isolated. Intermediates were separated from free DNA, finished DNA-filled capsids or empty procapsids by sucrose gradient sedimentation as in Example I. ATP, gp16, gp11/12 and gp9 were added to each of those fractions from the sucrose gradient that contained DNA-packaging intermediates, and assayed for the production of infectious virus. The production of infectious virus from completed DNA-filled particles was used as an indicator in testing the motor function in DNA packaging.

It was found that nanomotors turned off by γ-S-ATP were turned on again by ATP, since the DNA-packaging intermediates blocked by γ-S-ATP could be converted into matured infectious virion by the addition of gp16 and ATP as well as the neck protein gp11/gp12 and the tail protein gp9 (FIG. 14). The addition of ATP allowed the packaging of the entire viral DNA genome to be completed. The reactivation of the stationary nanomotor by ATP was further confirmed by direct observation of RNA rotation.

When EDTA was used to turn off the nanomotor, further analysis revealed that magnesium could turn it back on. However, a stationary nanomotor turned off by RNase was irreversible (FIG. 14).

The Nanomotor Could be Turned on and Off by gp16

As shown in Example I (FIGS. 4a and 4b), it was found that the candidate of the processive factor in this DNA-packaging motor is pRNA, while gp16 is a transient distributive factor in motor function. AptRNA functions as pRNA in the nanomotor. That is, aptRNA is an integrated solid part of the nanomotor, but gp16 is not. Without the addition of fresh gp16, not a single infectious virus particle was produced from the intermediates. This indicates that additional fresh gp16 is needed to complete assembly and that alternate gp16 molecules must have been involved in the DNA-packaging process. To repeat, gp16 is not a fixed solid part of the nanomotor, and the function of gp16 is contributive.

Packaged DNA was Released from the Protein Shell in the Presence of EDTA at Low pH or High Temperature

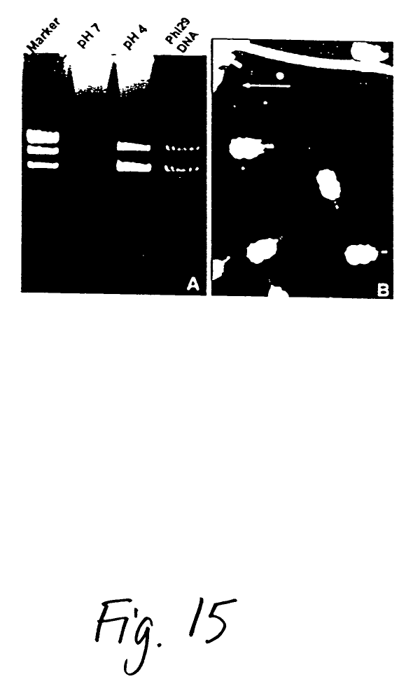

To determine the conditions for the reverse function of the nanomotor, the completed DNA-filled particles or infectious mature virions were treated with different pH, temperature and chemicals. The phi29 particles were contacted with buffers having pH 7 and pH 4 (lane c), then neutralized to pH 7, digested with the restriction enzyme EcoRI, and subjected to gel electrophoresis. FIG. 15 shows the pH 7 (lane b) and pH 4 (lane c) samples. It was found that in the presence of EDTA, the packaged DNA was released from the protein shell at pH 4 (FIG. 15), or at 75° C., but not at pH 7. DNA discharge is a passive motion process since no ATP is needed for such translocation.

Formation of the Ordered Structural Arrays

Due to the limitation in size, it is extremely difficult to detect, observe and build a structure using nano-parts. Formation of ordered structural arrays will greatly facilitate the application of nano-parts, such as in the manufacture of computer chips.

It was found that the in vitro synthesized nanomotor and motor parts formed a hexameric array, pentagonal particles and tetragonal arrays, depending on the condition and the number of parts present.

In 3M NaCl, the purified recombinant connector, composed of 12 subunits of gp10 protein, formed a well-ordered tetragonal array. Since the connector is a trapezoid-shaped cone, alternating facing-up and facing down arrangements facilitated the formation of the tetragonal array.

When six pRNAs were bound to the connector, the tetragonal arrays disappeared immediately. Rosettes containing five complexes composed of connector and hexameric RNA were formed with the RNA located at the center of the pentagonal rosette.

When an additional protein gp11 was added to the connector, a hexagonal array instead of tetragonal arrays was detected. The formation of the hexagonal array is due to the six-fold symmetry of the 12-subunit connector and the filling up of the narrow end of the trapezoid/cone-shape by the addition of six copies of pRNA and 12 copies of gp11 after an interaction with a hexameric RNA.

Up to 120 Nonspecific Bases Can be Extended from the 3′-End of aptRNA without Hindering the Function of the Nanomotor

To investigate whether additional burden can be imposed to the RNA, both the 3′ and 5′-ends of the aptRNA were extended with variable length. It was found that the 5′-end is not extendable, since a single base addition will render the RNA incompetent to drive the motor. However, up to 120 bases can be added to the 3′-end of the aptRNA without a significant interference of the motor function. Such addition includes the labeling with biotin, pCp, DIG and phosphate.

Discussion

The construction of a practical molecular shuttle requires a careful consideration of guiding the direction of motion, controlling the on-off status and speed, as well as the loading and unloading of cargo.

It was found that the direction of the DNA-packaging motor could be guided by adjusting the pH, the temperature or by the addition or omission of EDTA or ATP.

The nanomotor can be turned off by EDTA, γ-S-ATP, or RNase. Although the inactivation of the nanomotor by RNase was irreversible, the EDTA and γ-S-ATP effect can be negated by the addition of magnesium and/or ATP, respectively. This is an indication that the nanomotor inactivated by γ-S-ATP could be turned on by ATP, and that the nanomotor turned-off by EDTA could be turned on again by magnesium.

Gp16 can be used to control the running of the nanomotor, since a continuous supply of fresh gp16 is needed to keep the motor functioning. The control of ATP concentration, acting as a fuel supply, can serve as a means of controlling the speed of movement.

The loading process requires the coupling of cargo to the shuttle. The 120 bases extended from the 3′-end could serve as a tool for loading cargo. This can be achieved by attaching the cargo to a DNA that is complementary to the sequence at the 3′ end of the aptRNA. The formation of ordered structural arrays or particles will facilitate the construction of nanomachines. All this suggests that this DNA-packaging motor is a candidate component for use in the construction of nanodevices. This motor, expected to be a rotary machine with a mechanism similar to phi29 DNA-packaging motor that rotates in 12° increments, has been solved by mathematical simulation and direct observation.

### Example III

### Construction of phi29 DNA-Packaging RNA Monomers, Dimers, and Trimers with Variable Sizes and Shapes as Potential Parts for Nanodevices

Recently, DNA and RNA have been under extensive scrutiny with regard to their feasibility as parts in nanotechnology. The DNA-packaging motor of bacterial virus phi29 contains six copies of pRNA molecules, which together form a hexameric ring as an important part of the motor. This ring is formed via hand-in-hand interaction by Watson-Crick base pairing of four nucleotides from the left and right loops. This pRNA tends to form a circular ring by hand-in-hand contact even when in dimer or trimer form, thus implying that the pRNA structure is flexible. Stable dimers and trimers have been formed from the monomer unit in a protein-free environment with nearly 100% efficiency.

Dimers and trimers have been isolated by density gradient sedimentation or purified from native gel. Dimers and trimers were resistant to pH levels as low as 4 and as high as 10, to temperatures as low as −70° C. and as high as 80° C., and to high salt concentrations such as 2 M NaCl and 2 M MgCl₂. pRNA dimers or trimers with variable lengths were constructed. Seventy-five bases were found to be the central component in this formation. The elongation of RNA at the 3′ end up to 120 bases did not hinder their formation. RNA monomers, dimers, and trimers with variable lengths are potential parts for nanodevices (see Shu et al. J. Nanosci. Nanotech. 4(4): 295-302 (2003)).

Synthesis of pRNAs

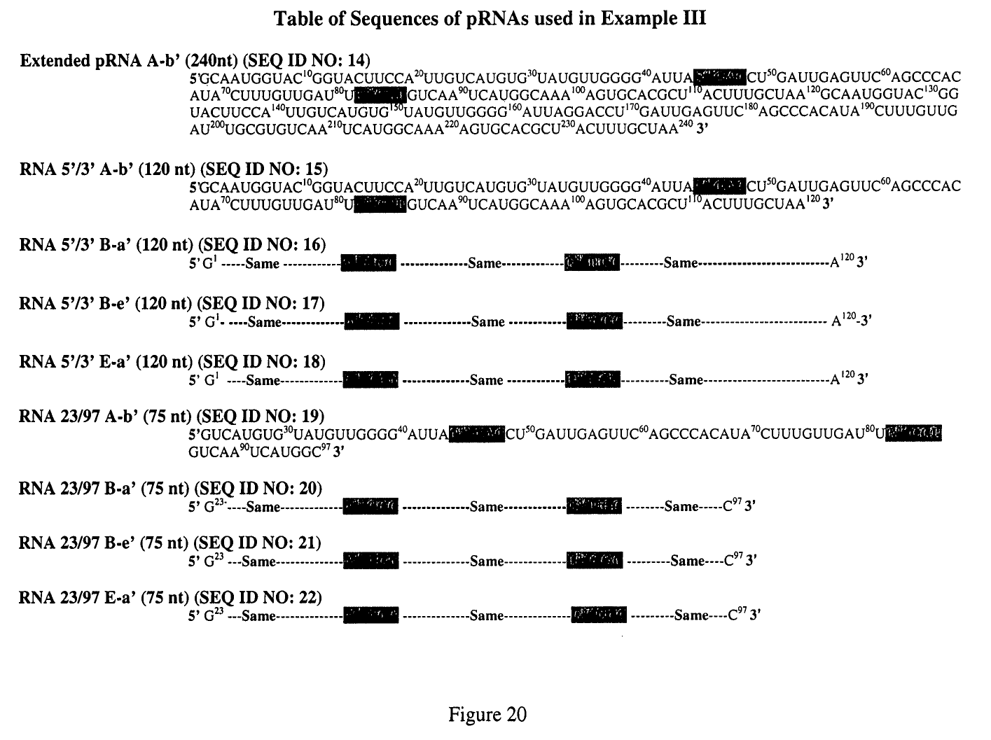

Synthesis and purification of full-length (120 base) and other pRNAs described herein and listed in FIG. 20 were performed substantially as described above in Examples I and II and also in C. L. Zhang et al., Virology 207: 442 (1995) and R. J. D. Reid et al., J. Biol. Chem. 269: 18656 (1994). pRNA nomenclature was reported in J. D. Reid et al., J. Biol. Chem. 269: 18656 (1994).

Specifically, the truncated 23/97 RNAs were synthesized by single-stranded DNA template transcription. Equal amounts of single-stranded DNA template and T7 top strand were mixed to form an annealed template (0.5 μM final) before being adding into the transcription mixture (which was composed of 4 mM NTPs, 40 mg/ml PEG 8000, 25 mM MgCl₂, 0.026 mg/ml T7 RNA polymerase, and 4 U/ml IPP (inorganic pyrophosphates), 0.77 mg/ml dithio-threitol, 0.25 mg/ml Spermidine, 0.05 mg/ml BSA and 40 mM Tris.Cl pH 8.0). After three hours of incubation at 37° C., the transcription reaction was stopped by 8M urea denaturing loading buffer.

Native TBM PAGE for Dimer and Trimer Detection

10% native polyacrylamide gels were prepared in TBM buffer (TBM: Tris 89 mM, boric acid 200 mM, MgCl₂ 5 mM, pH 7.6). Equal molar ratio of each of the pRNAs was applied to study the formation of dimers and trimers. After running at 4° C. for three hours, the RNA was visualized by ethidium bromide staining. Images were captured by an EAGLE EYE II system (Stratagene).

Isolation of Dimers and Trimers from Native PAGE

Tritiated pRNA A-b′ was mixed with B-a′ for dimers, and B-e′ plus E-a′ for trimers, and was subjected to electrophoresis in 10% native PAGE made in TBM. The pRNA dimer and trimer bands were excised from the gels and eluted overnight using the same TBM buffer at 4° C. These complexes were then either kept in TBM buffer at 4° C. for further use or frozen at −20° C.

Separation of pRNA Complexes by Sucrose Gradient Sedimentation

Linear 5-20% sucrose gradients were prepared in TBM buffer. The pRNA mixtures containing multimers were loaded onto the top of the gradient. To separate dimers from trimers, samples were spun in an SW55 rotor at 45,000 rpm for thirteen hours at 4° C. To separate dimers from monomers, samples were spun at 50,000 rpm for fourteen and one-half hours at 4° C. After sedimentation, fifteen-drop fractions were collected and subjected to scintillation counting.

In Vitro phi29 Virion Assembly Assay

10 μl of purified procapsids (0.013 μM) were dialyzed on a 0.025-μm VS filter against TBE for 15 minutes at ambient temperature. Various amounts of pRNAs, including monomers and dimers, were dissolved in 1.5 μl TMS buffer and then added to procapsids. Only a small volume was used to ensure a high concentration of pRNAs in the reaction. The mixtures were then dialyzed against TMS for another 30 minutes. The pRNA-enriched procapsids were mixed with gp16, DNA-gp3, and reaction buffer (10 mM ATP, 6 mM 2-mercaptoethanol, 3 mM spermidine in TMS) to complete the DNA packaging reaction. After 30 minutes, the neck, tail, and morphogenic proteins were added to the DNA packaging reactions to complete the assembly of infectious virions, which were then assayed by standard plaque formation (C. S. Lee et al., Virology 202: 1039 (1994)).

Results

Construction of Variable Length RNA Monomer, Dimer and Trimer

Uppercase letters are used to describe the right loop of the pRNA and lowercase to represent the left loop. The same letters in upper- and lowercase indicate complementary sequences, whereas different letters mean non-complementary loops. For example, pRNA 5′/3′(A-b′) represents a full-size pRNA with non-complementary right loop A (5′-G⁴⁵GAC) and left loop b′ (3′-U⁸⁵GCG) (FIG. 21-23).

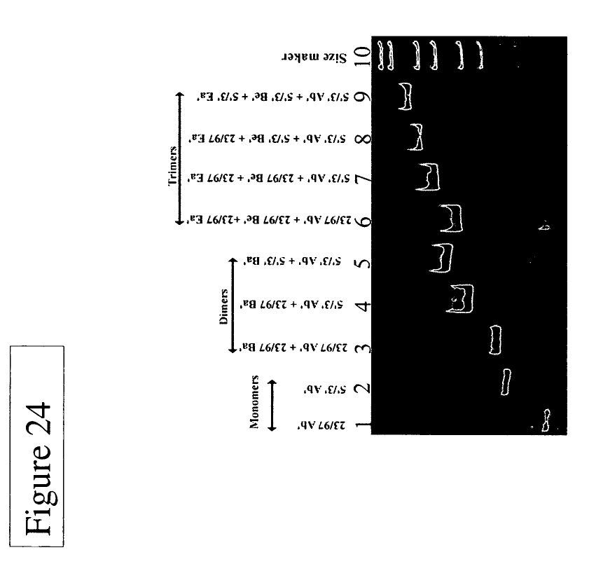

The monomer of full-size (5′/3′) and truncated (23/97) non-complementary pRNAs such as 5′/3′(A-b′), (B-a′), (B-e′) or (E-a′) and 23/97 (A-b′), (B-a′), (B-e′) or (E-a′) (FIG. 20) migrate faster in native gels (FIG. 24).

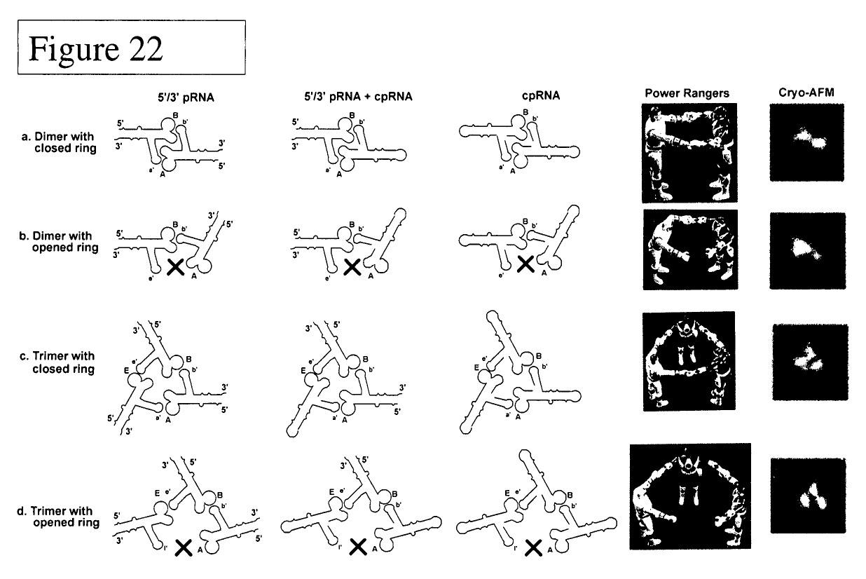

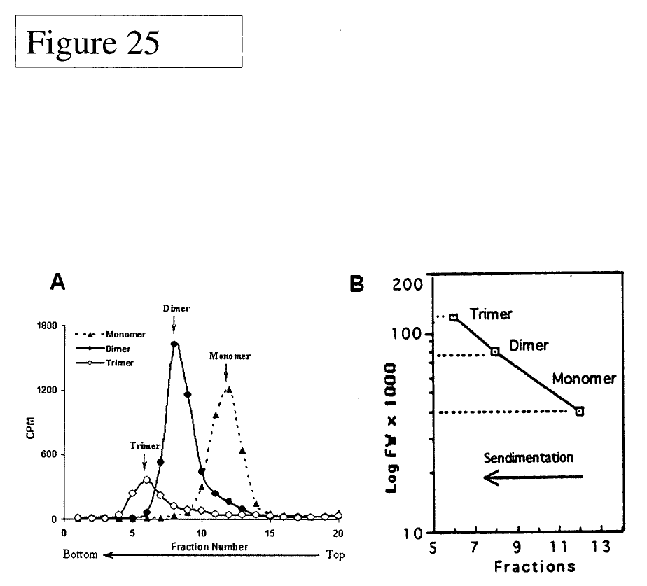

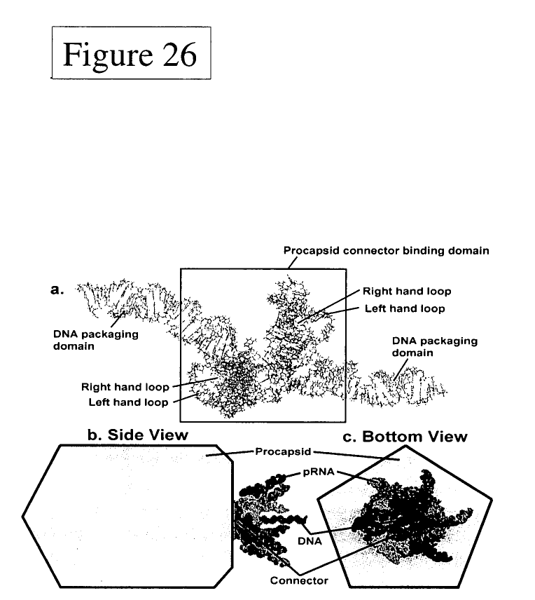

When the 5′/3′ or the 23/97 (A-b′) were mixed together with equal ratios of 5′/3′ or 23/97 (B-a′), RNAs shifted into slower migrating bands in native gels and proved to be dimers (FIG. 24, 25). The band of dimer with heterosized subunits was between that of the dimer 5′/3′(A-b′)-5′/3′(B-a′) and 23/97(A-b′)-23/97(B-a′). When three full-size or truncated RNAs with interlocking loops such as (A-b′)/(B-e′)/(E-a′) (in this example, RNA without prefix is full length 5′/3′ RNA, unless otherwise indicated) were mixed at equal molar concentration, a band in the native gel with a migration rate slower than that of a dimer was found and confirmed to be a trimer by sucrose gradient sedimentation (FIG. 25) and cryo-AFM (atomic force microscopy) (FIG. 22). Nucleotides 23-97 are the central components in the formation of both dimers and trimers. The ability to form dimers or trimers is not disturbed by 5′ or 3′ end truncation; one or two truncated pRNAs can be incorporated into dimers while one, two or three truncated pRNAs can be incorporated into trimers.

When analyzed by sucrose gradient sedimentation, [³H] pRNA monomers, dimers and trimers sedimented to fraction 12, 8 and 6, respectively (FIG. 25A). A plot of hypothetical molecular weight vs. the log of migration distance (the fractional number) in gradient showed a linear relationship (FIG. 25B). Thus the peaks of fraction 12, 8 and 6 could stand for monomer, dimer and trimer, respectively (FIG. 25A). The purified monomers, dimers and trimers are further confirmed by AFM imaging (FIG. 22).

pRNA has a Strong Tendency to form a Circular Ring by Hand-in-Hand Contact Regardless of Whether the pRNA Will Enter a Dimer, Trimer or Hexamer Form.

As reported previously (C. Chen et al., J. Biol. Chem. 275(23): 17510 (2000)) if a pRNA dimer or trimer contained a pair of non-complementary loops, the dimer or trimer was unstable. A closed ring could not be expected due to this faulty linkage. Results suggested that the formation of a closed ring by hand-in-hand interaction was required for the formation of a stable dimer or trimer complex in the solution (FIG. 21-23, 26). It also suggested that pRNA has a strong tendency to form a circular ring by hand-in-hand interaction, regardless of whether the pRNA is in dimer, trimer or hexameric form. It is obvious that the angles between the two loops in dimers and the two loops in trimers are different. Therefore, the pRNAs in dimer have adopted a different structure for intermolecular contact than the pRNAs in trimer, suggesting that the structure of pRNA is flexible and amendable.

Elongation of RNA at the 3′ End of the 120 Bases Did Not Hinder Dimer and Trimer Formation

Variable lengths of nucleotide sequences were extended from the 3′-end of the pRNA. The extended pRNA were tested for dimer and trimer formation. It was found that elongation of RNA at 3′ end of the 120 bases (FIG. 21) did not hinder the formation of dimer and trimer (data not shown). The C¹⁸C¹⁹A²⁰ bulge was found to be dispensable for both RNA dimer and trimer formation.

Inhibition by Truncated 23/97 RNA Dimer and Trimer in in Vitro Viral Assembly

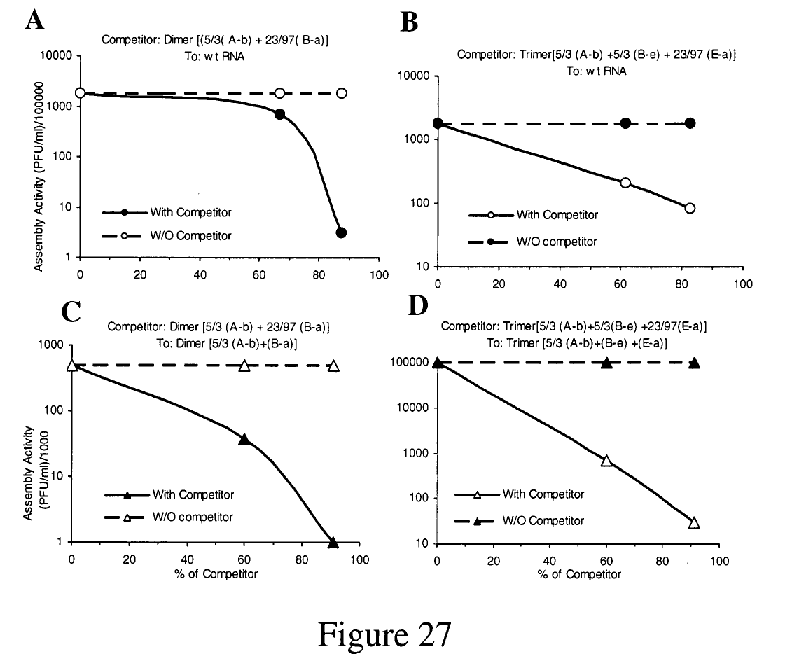

Truncated 23/97 RNA is inactive in DNA packaging. As discussed previously, the 23/97 segment RNA is a dimerization and trimerization unit. The inhibition study showed that the truncated dimer (A-b′)/(23/97B-a′) or the trimer (A-b′)/(B-e′)/(23/97E-a′) can partially inhibit the wild type monomer pRNA activity (FIG. 27). This means that a truncated dimer or trimer still has its correct biological folding. The reduced activity of wild type pRNA in the presence of a dead truncated dimer or trimer is due to the fact that the truncated dimer/trimer is still able to bind and occupy the RNA binding site in the procapsid in a competition nature. This competition binding nature was further confirmed by the fact that the truncated dimer (A-b′)/(23/97B-a′) and trimer (A-b′)/(B-e′)/(23/97E-a′) can strongly inhibit the plaque formation of wild type dimer (A-b)/(B-a) and trimer (A-b)/(B-e)/(E-a) (FIG. 27).

Testing the Stability of Dimer and Trimer by Ion Requirement, Salt Concentration, pH, Temperature, Electrophoresis and Sedimentation

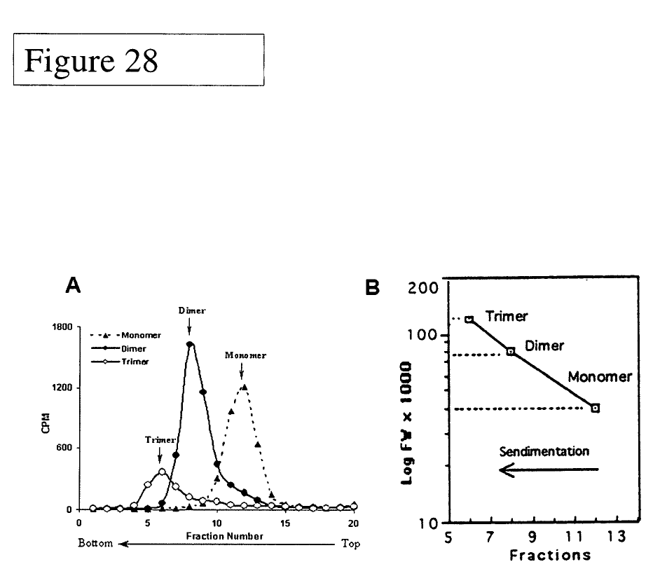

To detect the minimum ion concentration for pRNA oligomerization, equal amounts of tritiated (A-b′) and unlabeled (B-a′) were mixed and loaded onto the top of 5-20% sucrose gradient in TB buffer along with a variable amount of ions (FIG. 28A). At a concentration of 5 mM Mg⁺⁺, about 45% of tritiated (A-b′) centered at fraction 8, representing pRNA dimers. When the concentration was increased to 25 mM, about 90% of tritiated (A-b′) centered at the dimer position. While at a 1 mM or lower concentration, the tritiated (A-b′) remained as a monomer centered at fraction 2-4. The data indicated that at least 5 mM Mg⁺⁺ was required for detectable dimerization. The Mg⁺⁺ concentration requirement for dimer formation agrees with the data from polyacrylamide gel shift assay.

For circularly permuted cpRNAs (C. L. Zhang et al., Virology 207: 442 (1995)), the Mg⁺⁺ concentration required for 50% trimer formation was about 4 mM; while for pRNAs with wild type 5′/3′ ends, it was about 0.4 mM (C. Chen et al., J. Biol. Chem. 275(23): 17510 (2000)).

A minimal of 1M concentration of monovalent ions is needed for pRNA oligomerization, although as low as 5 mM of divalent ions is sufficient. Spermidine, a positively charged compound, can also stimulate oligomerization at a concentration of 5 mM, indicating that dimer or trimer formation is a result of a cation effect. CoCl₂ or NiCl₂ could not promote trimerization, while FeCl₂, ZnCl₂ or CdCl₂ caused the precipitation of pRNA (FIG. 28A). These data suggest that pRNAs could form oligomers in the presence of positively charged cations, including mono- or divalent cations, as well as spermidine. Formation of a multimeric ring is an intrinsic feature of pRNAs, and cations are a facilitator.

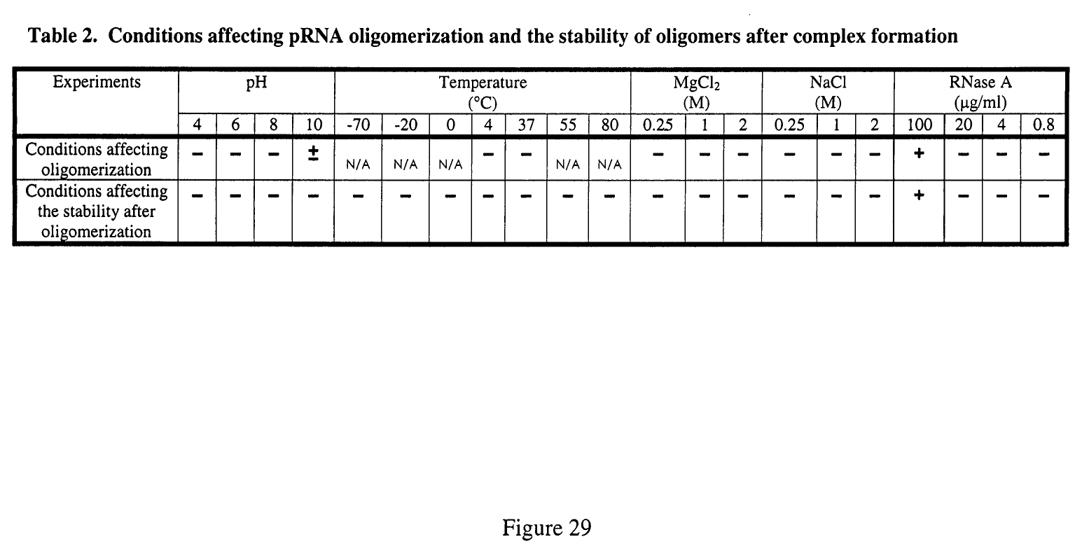

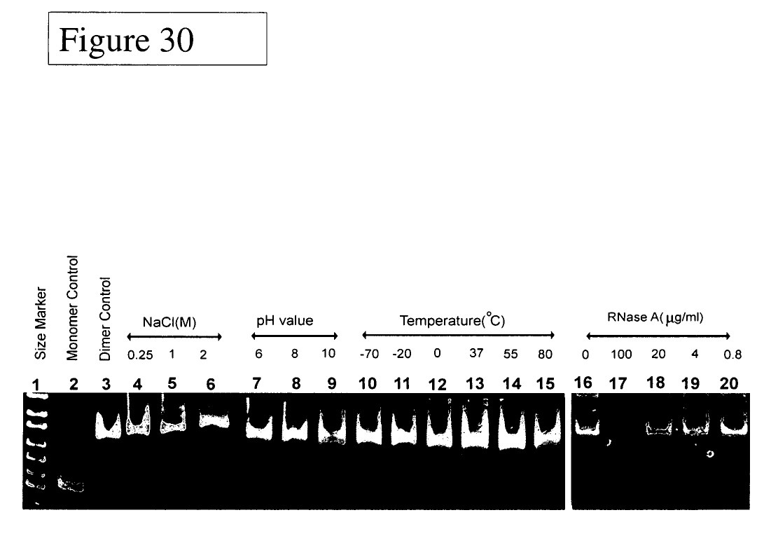

As shown in FIG. 25, before dimer or trimer purifications, stable dimers and trimers of pRNA were formed in a protein-free environment with nearly 100% efficiency. The dimer and trimer were found to be stable and could be isolated by either density gradient sedimentation or purification from native gel (FIG. 24-25). Dimers and trimers were resistant to a pH as low as 4 and as high as 10, a temperature as low as −70° C. and as high as 80° C. and a high salt concentration of 2M NaCl and 2M MgCl₂ (FIG. 29-30). 23/97 RNA is unstable when exposed to pH 10 buffer.

Discussion

A set of RNA molecules can be manipulated to form monomer, dimer, trimer and hexamer. The information governing the assembly of the diverse structure is encoded in a self-folded region with 74 nucleotides. Within this 74-base self-folded region, four bases in the left loop and another four bases in the right loop determine the formation of monomer, dimer, trimer or hexamer. These experiments reveal that the extension of the 3′-end of the pRNA does not interfere with its property of self-folding of the 74-base region. Thus, the 3′-end could have a similar function as the sticky end of DNA in building the branched structures. Gaining the advantage over DNA in the formation of helices and sticking end complementation, plus the intrinsic property of structure diversity, self-folding, and controllable length, this set of pRNA is a novel and unique way to build arrays or to serve as potential parts for nanodevices.

### Example IV

### Bottom-Up Assembly of RNA Arrays and Superstructures as Potential Parts in Nanotechnology

DNA has been extensively scrutinized for its feasibility for use in nanotechnology applications, but another natural building block, RNA, has been largely ignored. RNA can be manipulated to form versatile shapes, thus providing an element of adaptability to DNA nanotechnology, which is predominantly based upon a double-helical structure.

The DNA-packaging motor of bacterial virus phi29 contains six DNA-packaging pRNAs (pRNA), which together form a hexameric ring via loop/loop interaction. This pRNA can be redesigned to form a variety of structures and shapes, including twins, tetramers, rods, triangles, and arrays several microns in size via interaction of predetermined helical regions and loops.

In this Example, RNA array formation was found to require a defined nucleotide number for twisting of the interactive helix and a palindromic sequence. Such arrays were shown to be unusually stable and resistant to a wide range of temperatures, salt concentrations, and pH (see Shu et al., Nano Letters 4(9): 1717-1723 (2004)).

Synthesis of RNAs

The construction of pRNA and the synthesis, purification and nomenclature of bacterial virus phi29 pRNA have been reported previously (C. L. Zhang et al., Virology, 207: 442 (1995)).

Native or Denatured Polyacrylamide Gel for RNA Purification and the Detection of RNA Complexes and Arrays

After transcription, RNA was first purified from 8% denaturing polyacrylamide gel in the presence of 8 M urea. The pRNA monomer, twin (a twin is composed of two identical pRNAs bridged by a palindromic sequence at the 3′ end of pRNA), dimer and trimer bands were excised from the gels and eluted overnight using elution buffer (0.5M NH₄OAc, 0.1 mM EDTA, 0.1% SDS, and 0.5 mM MgCl₂ at 37° C.). The purified RNAs were used to construct dimers, trimers or arrays, which were analyzed by 5% to 8% native polyacrylamide prepared in TBM buffer (Tris 89 mM, boric acid 200 mM, MgCl₂ 5 mM, pH 7.6). The RNA was visualized by ethidium bromide staining. Images were captured by an EAGLE EYE II system (Stratagene). These complexes were then either kept in TBM buffer at 4° C. for further use or frozen at −20° C.

Separation of pRNA Complexes by Sucrose Gradient Sedimentation

Linear 5-20% sucrose gradients were prepared in TBM buffer. The RNA of multimers was loaded onto the top of the gradient. Samples were spun in an SW55 rotor at 40,000 rpm for twelve hours at 4° C. After sedimentation, twelve-drop fractions were collected and subjected to scintillation counting.

Cryo Atomic Force Microscopy (Cryo AFM) of pRNA Oligomers.

Oligomeric pRNA was purified from native polyacrylamide gels or sucrose gradient. To prepare the sample for cryo-AFM imaging of monomers, dimers and trimers, a piece of mica was freshly cleaved and soaked with spermidine. Excess spermidine was removed by repeated rinsing with deionized water. The pRNA sample (10 μg/ml) was applied to the mica, which had been pre-incubated with TBM buffer. After 30 seconds, the unbound pRNA was removed by rinsing with the same buffer. Before the sample was transferred to cryo-AFM for imaging, it was quickly rinsed with deionized water (<1 second) and the solution was removed with dry nitrogen within seconds. All cryo-AFM images were collected at 80 K. Scanlines were removed by an offline matching of the basal line. Calibration of the scanner was performed with mica and 1 μm dot matrix

To prepare the arrays, a 5 μL sample drop was spotted on freshly cleaved mica (Ted Pella, Inc.) and left to adsorb to the surface for 2 minutes. To remove buffer salts, 5-10 drops of doubly distilled water were placed on the mica, the drops were shaken off, and the sample was dried with compressed air. Imaging was performed under 2-propanol in a fluid cell on a NanoScope IIIa, using an NP-S oxide-sharpened silicon nitride probe (Veeco Probes).

Results

Construction of a Variety of pRNA Building Blocks to Build RNA Arrays or Superstructures.

Nanotechnology employs either the traditional top-down or the bottom-up approach. The “top-down” approach has been to design ever-smaller design features into existing technology whereas the “bottom-up” approach has attempted to build nanodevices one molecule at a time. Since the size of RNA ranges from the angstrom to the nanometer scale, the bottom-up approach could be reasonably applied to RNA in nanotechnological applications. Larger RNA complexes can be constructed from the following three building blocks: (a) monomer with intramolecularly self-complementary left and right loops, (b) monomer with non-complementary left and right loops for intermolecular interaction, (c) monomer with intermolecularly self-complementary left and right loops and palindromic 3′ ends.

Building blocks a and b have been described in Example III. The construction of building block c is depicted in FIGS. 31F & 31G.

Use of Monomeric Building Blocks to Construct RNA Twins, Tetramers, and Arrays

The use of monomer to construct dimers, and trimers has been discussed in Example III. These “designer” pRNA monomer derivatives, having predetermined left and right loops and palindromic ends, were used to build larger structures of RNA (FIG. 31-33).

When pRNA monomers with a non-complementary right loop (e.g., pRNA A-b′) and palindromic ends were purified from denaturing gel and renatured by the addition of 5 mM MgCl₂ into the solution, pRNA twins containing two identical monomers were formed. The formation of twins is highly efficient, approaching 100% even at concentrations as low as several ng/μl.

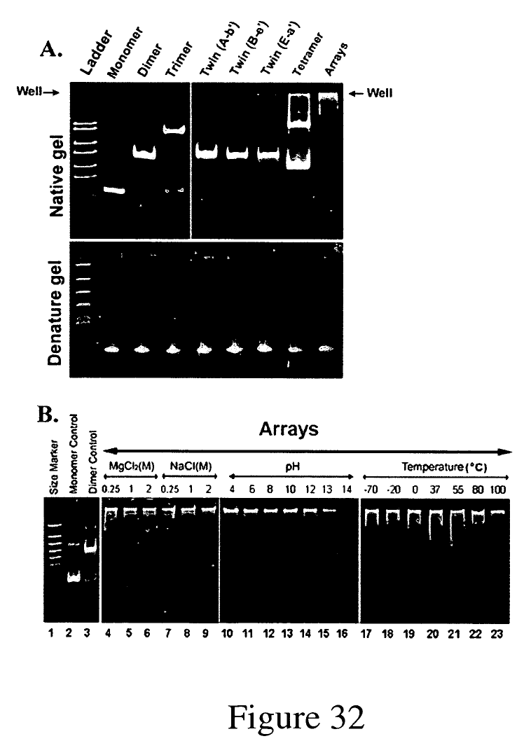

“Dimer” (FIG. 31E) refers to the complex composed of two different pRNA A-b′ and B-a′, while “twins” (FIG. 31G) are composed of two identical pRNAs bridged by a palindromic sequence at the 3′ end of pRNA. For example, twin A-b′ is composed of two identical pRNA A-b′ linked by the palindromic sequence “3′CGAUCG”. When two twins, for example, twin A-b′ and twin B-a′, were mixed in the presence of 10 mM MgCl₂, pRNA tetramers known as “double-twins” were produced (FIG. 32). When three twins, such as twin A-b′, twin B-e′, and twin E-a′, were mixed, pRNA began to grow into a micron-sized array by serial addition of the twins A-b′, B-e′, or E-a′. The arrays displayed as bundles as revealed by AFM (FIG. 34D), and grew to micron-scale, suggesting that each bundle contains hundreds of pRNA molecules.

When analyzed by 5% native polyacrylamide gel, pRNA monomers, dimers, trimers, twins, tetramers and arrays exhibited different migration rates (FIG. 32A). The array was too large to enter the gel and stayed trapped in the well (FIG. 32). It did not run into the gel even after electrophoresis for six hours in a 5% polyacrylamide gel.

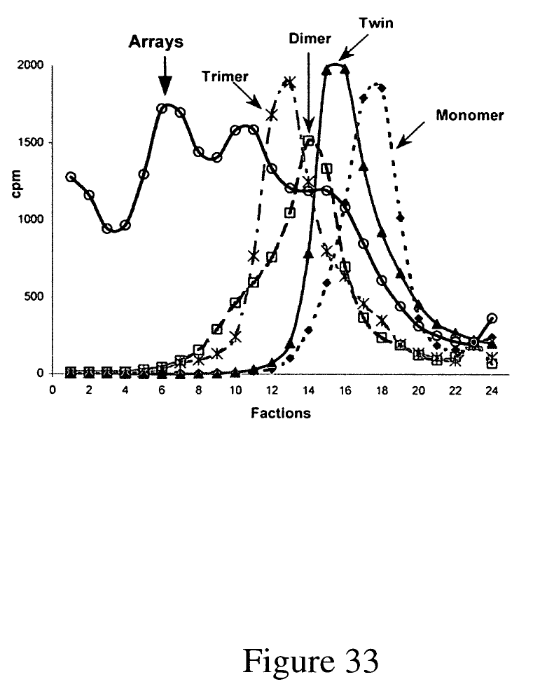

When analyzed by sucrose gradient sedimentation, [³H] pRNA monomers, twins, dimers and trimers sedimented to fractions 18, 16, 14 and 13, respectively (FIG. 33). A plot of hypothetical molecular weight vs. the log of migration distance (the fractional number) in longer sedimentation gradients revealed a linear relationship among monomers, dimers and trimers (data not shown). The purified monomers, dimers and trimers have been further confirmed by AFM imaging (FIG. 34). The twins were sedimented to a position similar to the dimers. The array exhibited a rapid sedimentation rate that was close to that for a DNA with more than ten thousand base pairs.

The Effect of the Twisting Angle and the Length of the Interacting Helical Region on Array Formation

It is expected that arrays will grow by nucleation from one building block of pRNA and can grow in solution with three-dimensional extensions. Therefore, the twisting angle of the extending area between the two building blocks is important to proper array growth. The 5′/3′ paired helical junction region composed of nucleotides 1-28 and 92-117 (FIG. 31) and will be important in governing the extending direction of the next RNA building block. It would be desirable to restrict the joining of the two helical regions to an odd number of half-turns (180°). Since each helical turn of RNA is composed of eleven nucleotides, 50 nucleotides (FIG. 31) will result in nine half-turns (4.5 turns). When the 50 nucleotides were used as the initial design in array formation, array extension continued successfully (FIG. 34D) and the formation of arrays was detected using this parameter. To test the effect of twisting angle and length of the interacting helical region in array formation, one nucleotide was eliminated from the helical region, resulting in a helical junction region with only 48 nucleotides (27 for each monomer, with 6 bases to be paired). It was found that arrays would not form using this truncated pRNA, since 48 nucleotides generates only 8.7 half-turns.

Effect of the Sequence of the Interacting Left and Right Loops on Array Stability.

It is expected that the interactive left and right loops play an important role in the growth and extension of arrays. pRNA building blocks with different loop sequences were constructed and tested. The stability of arrays was tested by hour-long electrophoresis in polyacrylamide gel at elevated temperatures. It was found that arrays from the building blocks with the loop A-a′ were much more stable than pRNA with loop I-i′ (1, 5′AACC; i′, 3′UUGG), suggesting that such loops play a critical role in array stability.

Determination of the Effect of Salt, pH and Temperature on pRNA Complex Formation and Stability.

The minimum ion concentration requirement for pRNA array formation was determined by both polyacrylamide gel shift assay and sucrose gradient sedimentation. It was found that although 5 mM of divalent ions such as MgCl₂, CaCl₂ and MnCl₂ were needed, a minimum of 1 M of monovalent ions such as NaCl alone was needed for the formation of pRNA arrays. The arrays were resistant to pH values as low as 4 and as high as 12, temperatures as low as −70° C. and as high as 100° C., and high salt concentrations of 2 M NaCl and 2 M MgCl₂. At pH13, only portions of the arrays were degraded (FIG. 32, lane 15). This indicates that pRNA arrays are much more stable than regular 120-nucleotide RNA, which has an unfolding T_{M} between 50-70° and is sensitive to pH values higher than 11. Such stability is credited to the tightly folded pRNA structure and to the intertwining of the RNA molecule in the arrays.

Discussion

RNA arrays can be constructed through the use of pRNA twins, dimers, trimers, or hexamers as building blocks (FIG. 34). Palindromic sequences were introduced into the 3′ end of the pRNA, and can serve as links for bridging and intermolecular interaction. The left and right loops can be used to aid array growth by continuous extension via loop/loop intermolecular interaction. Gel electrophoresis and AFM images revealed that interaction of the palindromic sequences with the right and left loops causes the formation of pRNA arrays composed of a huge number of twins (FIG. 34D).

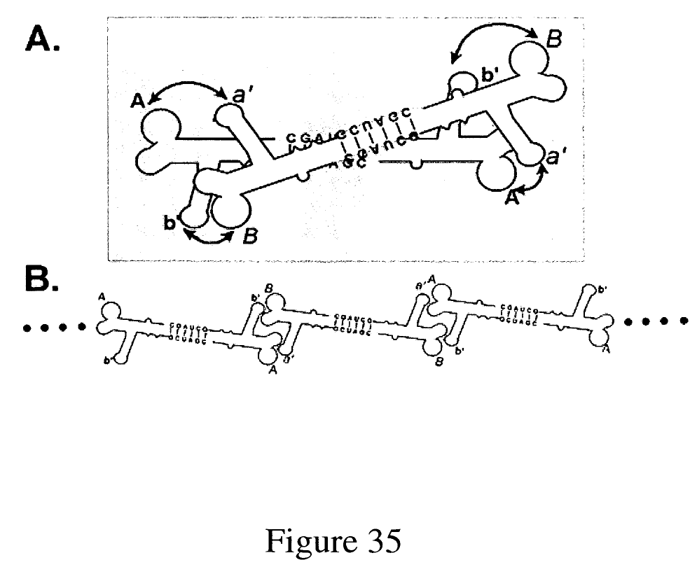

Two alternate assembly pathways were observed after mixing two different twins with intermolecularly compatible loop sequences. This may be attributed to the structural flexibility inherent to pRNA. The formation of tetramers indicates that the two twins were able to form a closed circular structure (FIG. 35), and since the four domains for intermolecular interaction were partnered, assembly ceased. This pathway competed with the formation of chains (FIG. 35) or possibly larger circles of twins, which assembled to a size above the polyacrylamide gel separation limit (FIG. 33, the tetramer lane). As previously reported, the UUU bulge at the three-way junction serves as a hinge to provide for the flexibility of pRNA, which enables the dimerization of twins and is also evident in the assembly into hexamers (C. Chen et al., J. Biol. Chem. 275(23): 17510-17516 (2000)) from dimers via hand-in-hand interactions (C. Chen et al., RNA 5: 805-818 (1999), P. Guo et al., Mol. Cell. 2: 149-155 (1998)). In dimers, each pRNA monomer subunit only holds the hands of one additional pRNA. In hexamers, however, each pRNA monomer subunit holds the hands of two additional pRNAs (FIG. 15 in S. Hoeprich et al., J. Biol. Chem. 277(23): 20794-20803 (2002)). This may at first seem paradoxical given the hand interactions of dimers and hexamers, but such interaction can be accounted for if the conformational change of pRNA and the presence of a hinge at the three-way junction are considered. The flexibility displayed by pRNA on the dimer-to-hexamer assembly pathway may also be an essential intrinsic feature of pRNA, enabling its function in DNA translocation (S. Hoeprich et al., J. Biol. Chem. 277(23): 20794-20803 (2002)). In dimer-to-hexamer assembly, connector binding of a closed dimer is associated with breaking up one of the two hand-in-hand interactions and a dramatic change in the relative orientation of the two pRNA monomers, which requires a reorientation of the binding loops. The dimer of twin formation may be enabled by a similar structural transition involving a loop hinge.

The rate of sedimentation is generally dependent not only on molecular weight but also on the shape of the molecule. Dimers are more compact than twins, which explain why dimers migrate more quickly in sucrose gradient. At any movement, the extension of arrays can terminate and lead to the abortive smaller structure. This might explain the broad peak and multiple curves in sucrose gradient sedimentation. Such a broad peak and multiple curves could not be separated by polyacrylamide gel since molecules more than 1000 nucleotides are beyond the resolution limit of polyacrylamide gel.

As expected, the twisting angle between the two loop regions in a twin had a major effect on array formation. Deletion of two bases from the stem of the twin is expected to change the angle between the two loop regions by about 65.5°. In the twins that gave rise to extended arrays, the two loop regions were roughly in a planar alignment.

The complete disclosure of all patents, patent applications, and publications, and electronically available material (including, for example, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

1. A molecular nanomotor comprising, as structural components:
a gp10 connector protein component;
a gp8 capsid protein component; and
a non-naturally occurring pRNA component;
wherein the structural components are associated with one another to form a nanoscale structure that effects translocation of a polynucleotide in the presence of a gp16 protein, ATP and Mg⁺⁺.

2. The molecular nanomotor of claim 1 wherein the non-naturally occurring pRNA is one that folds into a structure similar to that of naturally occurring phi29 pRNA (SEQ ID NO: 2).

3. The molecular nanomotor of claim 1 further comprising a protein gp7.

4. The molecular nanomotor of claim 1 wherein the translocation activity can be reversibly stopped by contacting the nanomotor with a metal chelating agent, contacting the nanomotor with a nonhydrolyzable ATP analogue, or depriving the nanomotor of a source of gp16 protein, ATP or Mg⁺⁺.

5. The molecular nanomotor of claim 1 wherein the translocation activity can be reversibly stopped by contacting the nanomotor with γ-S-ATP.

6. The molecular nanomotor of claim 1 wherein translocation activity can be reversibly stopped by contacting the nanomotor with EDTA.

7. An isolated molecular nanomotor comprising, as structural components:
a connector protein component;
a capsid protein component; and
a pRNA component;
wherein the structural components are associated with one another to form a nanoscale structure that effects translocation of a polynucleotide in the presence of ATP and Mg⁺⁺, and wherein the pRNA binds ATP and drives the rotational motion of the nanomotor.

8. The isolated molecular nanomotor of claim 7 wherein the pRNA is selected from the group consisting of SF5 pRNA (SEQ ID NO: 5), B103 pRNA (SEQ ID NO: 6), M2/NF pRNA (SEQ ID NO: 7) and GA1 pRNA (SEQ ID NO: 8).

9. The isolated molecular nanomotor of claim 7 wherein the pRNA folds into a structure similar to that of naturally occurring pRNA from SF5, B103, M2/NF or GA1.

10. The isolated molecular nanometer of claim 7 wherein the pRNA is a non-naturally occurring pRNA.

11. The molecular nanomotor of claim 7 wherein the translocation activity can be reversibly stopped by contacting the nanomotor with a metal chelating agent, contacting the nanomotor with a nonhydrolyzable ATP analogue, or depriving the nanomotor of a source of gp16 protein, ATP or Mg⁺⁺.

12. The molecular nanomotor of claim 7 wherein the translocation activity can be reversibly stopped by contacting the nanomotor with γ-S-ATP.

13. The molecular nanomotor of claim 7 wherein translocation activity can be reversibly stopped by contacting the nanomotor with EDTA.

14. A method for translocating a polynucleotide comprising:
providing a molecular nanomotor having a nanoscale structure according to claim 1; and
contacting the nanoscale structure with a gp16 protein, ATP and Mg⁺⁺ under conditions to translocate the polynucleotide.

15. The method of claim 14 further comprising contacting the nanoscale structure with a chelating agent or a nonhydrolyzable ATP analogue to reversibly stop translocation of the polynucleotide.

16. The method of claim 15 wherein the chelating agent is EDTA.

17. The method of claim 15 wherein the nonhydrolyzable ATP analogue is γ-S-ATP.

18. A method for translocating a polynucleotide comprising:
providing a molecular nanomotor having a nanoscale structure according to claim 5; and
contacting the nanoscale structure with a gp16 protein, ATP and Mg⁺⁺ under conditions to translocate the polynucleotide.

19. The method of claim 18 further comprising contacting the nanoscale structure with a chelating agent or a nonhydrolyzable ATP analogue to reversibly stop translocation of the polynucleotide.

20. The method of claim 19 wherein the chelating agent is EDTA.

21. The method of claim 20 wherein the nonhydrolyzable ATP analogue is γ-S-ATP.

22. The molecular nanomotor of claim 1 or 7 wherein the pRNA comprises bases 23 through 97 of phi29 pRNA.

23. The molecular nanomotor of claim 1 or 7 wherein the pRNA comprises a primary sequence that yields the same three-dimensional structure as bases 23 through 97 of phi29 pRNA, said primary sequence containing one or more base pairs that covary in relation to the phi29 pRNA primary sequence.

24. The molecular nanomotor of claim 1 or 7 comprising at least one pRNA comprising a 3′ pRNA extension region.

25. The molecular nanomotor of claim 24 wherein the 3′ extension region comprises a capture region.

26. The molecular nanomotor of claim 25 wherein the 3′ capture region hybridizes to a polynucleotide.

27. The molecular nanomotor of claim 24 wherein the 3′ extension region comprises a reactive group for attachment to a substrate.

28. The method of claim 14 or 18 wherein the gp16 protein comprises an N-terminal extension region.

29. The method of claim 14 or 18 wherein the polynucleotide is linked to a molecular cargo, and wherein the molecular cargo is also translocated.

30. A method for sorting polynucleotides comprising:
providing a molecular sorting device comprising the molecular nanomotor of claim 1 or 7 comprising at least one pRNA comprising a 3′ pRNA extension region comprising a capture region that hybridizes to a polynucleotide; and
contacting the molecular sorting device with a mixture of polynucleotides under conditions that permit selective hybridization of the polynucleotide to the 3′ extension region followed by translocation of the selected polynucleotide.

31. A microarray comprising a multiplicity of pRNA molecules.

32. The microarray of claim 31 wherein the pRNA molecules are naturally occurring or non-naturally occurring.

33. The microarray of claim 31 wherein at least a portion of the pRNA molecules have a three-dimensional structure which is the same as that formed by bases 23 through 97 of phi29 pRNA.

34. The microarray of claim 33 wherein at least a portion of the pRNA molecules comprise bases 23 through 97 of phi29 pRNA.

35. The microarray of claim 33 wherein the primary sequence of at least a portion of the pRNA molecules contains one or more base pairs that covary in relation to the phi29 pRNA primary sequence.

36. The microarray of claim 31 comprising at least one pRNA oligomer selected from the group consisting of a dimer, trimer, tetramer, hexamer, twin and double twin.

37. The microarray of claim 31 wherein at least a portion of the pRNA molecules comprise right and left loops; and wherein the right or left loop, or both, comprise an intramolecularly or intermolecularly complementary nucleotide sequence.

38. The microarray of claim 31 wherein at least a portion of the pRNA molecules comprise palindromic 3′ and 5′ ends.

39. The microarray of claim 31 wherein at least a portion of the pRNA molecules comprise circularly permuted pRNA (cpRNA).

40. The microarray of claim 31 comprising pRNA monomers.

41. The microarray of claim 40 wherein at least a portion of the pRNA monomers comprise a helical junction region resulting in an odd number of half-turns.

42. The microarray of claim 41 wherein the odd number of half turns extends the area between the two monomers to allow for continued array growth.

43. The microarray of claim 31 wherein at least a portion of the pRNA molecules form a shape selected from a checkmark, a rod, a triangle, a bundle, a spiral and a hairpin.

44. The microarray of claim 31 wherein at least a portion of the pRNA molecules comprise a 3′ extension region.

45. The microarray of claim 44 wherein the 3′ extension region comprises a capture region.

46. The microarray of claim 45 wherein the 3′ capture region hybridizes to a polynucleotide.

47. The microarray of claim 44 wherein the 3′ extension region comprises a reactive group for attachment to a substrate.

48. The microarray of claim 31 which forms a lattice or scaffolding.

49. The microarray of claim 31 comprising a two-dimensional array.

50. The microarray of claim 31 comprising a three-dimensional array

51. A nanoscale device comprising the molecular nanomotor of claim 1 or 7.

52. A nanoscale device comprising the microarray of claim 31.

53. A nanoscale device comprising lattice or scaffolding comprising a multiplicity of pRNA molecules.
